(19) United States
(12) Patent Application Publication
(10) Pub. No.: US 2023/0365941 A1
(43) Pub. Date: Nov. 16, 2023

(54) ORGANOID RECOMBINATION

(71) Applicant: CHILDREN'S HOSPITAL MEDICAL CENTER, Cincinnati, OH (US)

(72) Inventors: Michael A. Helmrath, Cincinnati, OH (US); Simon Vales, Vertou (FR); Nambirajan Sundaram, Mason, OH (US); Akaljot Singh, Cincinnati, OH (US); Nicole Brown, Blue Ash, OH (US)

(21) Appl. No.: 18/029,863

(22) PCT Filed: Sep. 29, 2021
(86) PCT No.: PCT/US2021/052728

§ 371 Date: Mar. 31, 2023

Related U.S. Application Data
(60) Provisional application No. 63/086,866, filed on Oct. 2, 2020.

(51) Int. Cl. C12N 5/071; C12N 5/0775

(52) U.S. Cl. CPC C12N 5/0697; C12N 5/0679; C12N 5/0668; C12N 2506/45; C12N 2533/90; C12N 2513/00; C12N 2501/11

(57) ABSTRACT

Disclosed herein are organoid compositions having heterogeneous combinations of epithelial and mesenchymal components, and methods of making the same by dissociating and recombining the epithelial and mesenchymal components from different sources. These epithelial and mesenchymal components can be derived from the same or different cell type or organoid type. These organoid compositions may exhibit advantageous properties, for example, enhanced in vivo engraftment.

### STATEMENT REGARDING FEDERALLY SPONSORED R&D

This invention was made with government support under DK103117 awarded by the National Institutes of Health. The government has certain rights to the invention.

### FIELD OF THE INVENTION

Aspects of the present disclosure relate generally to organoid compositions and methods of making the same involving the dissociation and combination of epithelial and mesenchymal components of cell compositions such as organoids.

### BACKGROUND

Organoids, particularly those derived from pluripotent stem cells, closely resemble in vivo tissue and have been shown to have great potential in many applications such as drug screening, transplantation, and personalized medicine. However, organoids produced by existing methods are still limited in certain properties compared to in vivo tissue, for example, the ratio of epithelial and mesenchymal lineages found in the differentiated organoid. Applicant's initial work identified methods that enable pluripotent stem cells to differentiate into definitive endoderm and mesenchyme that support in vivo engraftment. These patterned structures reflect the proximal small bowel and are referred to as Human Intestinal Organoids (HIO). Recent methods to pattern foregut (e.g. human gastric organoids [HGO]) and hindgut (e.g. human colonic organoids [HCO]) in vitro result in heterogenicity in epithelial to mesenchymal ratios. There is a present need for organoids having improved epithelial/mesenchymal ratios and morphologies, and methods of making the same.

### SUMMARY

One aspect of the present disclosure are methods of producing a composite organoid. In some embodiments, the methods comprise obtaining mono-dissociated mesenchymal cells isolated from one or more organoids, obtaining an epithelial structure isolated from an organoid or enteroid, combining the mono-dissociated mesenchymal cells and the epithelial structure, and culturing the combined mono-dissociated mesenchymal cells and the epithelial structure to form the composite organoid. In some embodiments, the mono-dissociated mesenchymal cells, or the epithelial structure, or both, are isolated by mechanical dissociation and filtration. In some embodiments, the mono-dissociated mesenchymal cells and the epithelial structure are combined by centrifugation. In some embodiments, the number of mesenchymal cells in the composite organoid is greater than the original number of mesenchymal cells of the organoid or enteroid from which the epithelial structure is isolated, such that the composite organoid has an enriched mesenchyme. In some embodiments, 1) the one or more organoids from which the mono-dissociated mesenchymal cells are isolated and 2) the organoid or enteroid from which the epithelial structure is isolated each comprises a tissue type selected from an esophageal, gastric, hepatic, intestinal, or colonic tissue type, or any combination thereof. In some embodiments, the tissue type of the one or more organoids and the tissue type of the organoid or enteroid are different. In some embodiments, the tissue type of the one or more organoids from which the mono-dissociated mesenchymal cells are isolated and the tissue type of the organoid or enteroid from which the epithelial structure is isolated are different, where no repatterning of the epithelial structure by the mono-dissociated mesenchymal cells occurs, such that the epithelium of the composite organoid maintains the tissue type of the organoid or enteroid from which the epithelial structure is isolated. In some embodiments, no repatterning of the epithelial structure occurs when the epithelial structure is derived from an organoid that is at least 14, 15, 16, 17, 18, 19, 20, 21, 22, 23, 24, 25, 26, 27, 28, 29, or 30 days old, or an organoid that is between 14-30, 15-30, 18-30, 15-20, or 15-25 days old. In some embodiments, no repatterning of the epithelial structure occurs when the epithelial structure is derived from an enteroid. In some embodiments, the enteroid is derived from adult tissue. In some embodiments, the tissue type of the one or more organoids from which the mono-dissociated mesenchymal cells are isolated and the tissue type of the organoid or enteroid from which the epithelial structure is isolated are different, where repatterning of the epithelial structure by the mono-dissociated mesenchymal cells occurs, such that the epithelium of the composite organoid exhibits properties of the tissue type of the one or more organoids from which the mono-dissociated mesenchymal cells are isolated. In some embodiments, repatterning of the epithelial structure by the mono-dissociated mesenchymal cells may occur when the epithelial structure is derived from an organoid that is no more than 8, 9, 10, 11, 12, or 13 days old, or an organoid that is between 8-13, 8-10, or 10-13 days old. In some embodiments, enteroids are derived from adult tissue with well-defined patterning, and epithelial structures derived from enteroids maintain their tissue type even when recombined with the mono-dissociated mesenchymal cells. In some embodiments, the tissue type of the one or more organoids and the tissue type of the organoid or enteroid are the same. In some embodiments, the one or more organoids and the organoid or enteroid each comprises only one of the esophageal, gastric, hepatic, intestinal, or colonic tissue type, such that the resultant composite organoid is a homogenous organoid comprising one tissue type. In some embodiments, the one or more organoids is derived from pluripotent stem cells (PSCs) from a first subject, and the organoid or enteroid is derived from PSCs or isolated from gastrointestinal tissue from a second subject. In some embodiments, the first subject and the second subject are mammals. In some embodiments, the first subject and the second subject are humans. In some embodiments, the first subject and the second subject are the same individual.

In some embodiments of any of the methods disclosed herein, the methods further comprise transplanting the composite organoid to a recipient subject. In some embodiments, the recipient subject is the first subject or the second subject. In some embodiments, the composite organoid exhibits greater engraftment and growth in the recipient subject compared to a comparable non-composite organoid or enteroid.

Also disclosed herein are composite organoids. The composite organoids comprise a mesenchyme comprising mesenchymal cells isolated as mono-dissociated cells from a first organoid and an epithelium comprising epithelial cells isolated as an epithelial structure from a second organoid or enteroid. In some embodiments, the ratio of the number of mesenchymal cells to epithelial cells in the composite organoid is greater than that of the second organoid or enteroid. In some embodiments, the first organoid and second organoid or enteroid each comprises a tissue type selected from an esophageal, gastric, hepatic, intestinal, or colonic tissue type, or any combination thereof. In some embodiments, the tissue type of the first organoid and the tissue type of the second organoid or enteroid is the same. In some embodiments, the tissue type of the first organoid and the tissue type of the second organoid or enteroid is different. In some embodiments, the composite organoid is the organoid produced by any one of the methods disclosed herein.

Also disclosed herein are composite organoids comprising a mesenchyme comprising a first tissue type selected from an esophageal, gastric, hepatic, intestinal, or colonic tissue type, or any combination thereof, and an epithelium comprising a second tissue type selected from an esophageal, gastric, hepatic, intestinal, or colonic tissue type, or any combination thereof. In some embodiments, the first tissue type and second tissue type have at least one difference in tissue types.

Also disclosed herein is the use of any one of the organoids disclosed herein for treating a gastrointestinal malady in a subject in need thereof.

Also disclosed herein are methods of screening for a candidate therapeutic, comprising contacting any one of the organoids disclosed herein with the candidate therapeutic and determining the effect of the candidate therapeutic on the organoid.

Embodiments of the present disclosure provided herein are described by way of the following numbered alternatives:
1. A method of producing a composite gastrointestinal organoid, comprising:
a) isolating mono-dissociated mesenchymal cells from one or more gastrointestinal organoids of a first tissue type;
b) isolating an epithelial structure from a gastrointestinal organoid or enteroid of a second tissue type;
c) combining the mono-dissociated mesenchymal cells and the epithelial structure; and
d) culturing the combined mono-dissociated mesenchymal cells and the epithelial structure to form the composite gastrointestinal organoid;
wherein the number of mono-dissociated mesenchymal cells is greater than the original number of mesenchymal cells of the gastrointestinal organoid of the second type and the composite gastrointestinal organoid has an enriched mesenchyme.
2. The method of alternative 1, wherein the mono-dissociated mesenchymal cells, or the epithelial structure, or both, are isolated by mechanical dissociation and filtration.
3. The method of any one of the preceding alternatives, wherein the mono-dissociated mesenchymal cells and the epithelial structure are combined by centrifugation.
4. The method of any one of the preceding alternatives, wherein the first tissue type and the second tissue type each are independently selected from the group of tissue types consisting of an esophageal, gastric, hepatic, intestinal, or colonic, or a tissue type that is a combination of any of the preceding.
5. The method of any one of the preceding alternatives, wherein the first tissue type and the second tissue type are the same, and the tissue type of the resulting composite gastrointestinal organoid is a homogenous organoid.
6. The method of any one of alternatives 1-4, wherein the first tissue type and the second tissue type are different, and the tissue type of the resulting composite gastrointestinal organoid is a heterogeneous organoid.
7. The method of any one of the preceding alternatives, wherein the one or more gastrointestinal organoids of the first type is derived from pluripotent stem cells (PSCs) from a first subject, and wherein the gastrointestinal organoid or enteroid is derived from PSCs or isolated from gastrointestinal tissue from a second subject.
8. The method of alternative 7, wherein the first subject and the second subject are mammals.
9. The method of alternative 7 or 8, wherein the first subject and the second subject are humans.
10. The method of any one of alternatives 7-9, wherein the first subject and the second subject are the same individual.
11. The method of any one of the preceding alternatives, further comprising transplanting the composite gastrointestinal organoid to a recipient subject.
12. The method of alternative 11, wherein the recipient subject is the first subject or the second subject.
13. The method of alternative 11 or 12, wherein the composite gastrointestinal organoid exhibits greater engraftment and growth in the recipient subject compared to a comparable non-composite organoid of the one or more gastrointestinal organoids of the first type, or a comparable non-composite organoid or enteroid of the gastrointestinal organoid or enteroid of the second type, or both.
14. A composite gastrointestinal organoid, comprising: a mesenchyme comprising cells from a gastrointestinal organoid of a first source; and an epithelium from a gastrointestinal organoid or enteroid of a second source;
wherein the ratio of the number of mesenchymal cells to epithelial cells in the composite gastrointestinal organoid is greater than that of the organoid or enteroid of the second type; and/or wherein the tissue type of the first source is different from the tissue type of the second source.
15. The gastrointestinal organoid of alternative 14, wherein the first gastrointestinal type and the second gastrointestinal type each comprises an esophageal type, gastric type, hepatic type, intestinal type, or colonic type, or any combination thereof.
16. The gastrointestinal organoid of alternative 14 or 15, wherein the first gastrointestinal type and the second gastrointestinal type is the same, and the composite gastrointestinal organoid is a homogenous organoid.
17. The gastrointestinal organoid of alternative 14 or 15, wherein the first gastrointestinal type and the second gastrointestinal type is different, and the composite gastrointestinal organoid is a heterogeneous organoid.
18. A composite gastrointestinal organoid produced by the method of any one of alternatives 1-13.
19. The gastrointestinal organoid of any one of alternatives 14-18 for use in treating a gastrointestinal malady in a subject in need thereof.
20. A method of producing a composite organoid, comprising:
a) isolating mono-dissociated mesenchymal cells from one or more organoids;
b) isolating an epithelial structure from an organoid or enteroid;
c) combining the mono-dissociated mesenchymal cells and the epithelial structure; and
d) culturing the combined mono-dissociated mesenchymal cells and the epithelial structure to form the composite organoid.
21. The method of alternative 20, wherein the mono-dissociated mesenchymal cells, or the epithelial structure, or both, are isolated by mechanical dissociation and filtration.
22. The method of any one of the preceding alternatives, wherein the mono-dissociated mesenchymal cells and the epithelial structure are combined by centrifugation.
23. The method of any one of the preceding alternatives, wherein the number of mesenchymal cells in the composite organoid is greater than the original number of mesenchymal cells of the organoid or enteroid from which the epithelial structure is isolated, such that the composite organoid has an enriched mesenchyme.
24. The method of any one of the preceding alternatives, wherein the one or more organoids from which the mono-dissociated mesenchymal cells are isolated and the organoid or enteroid from which the epithelial structure is isolated each comprises a tissue type selected from an esophageal, gastric, hepatic, intestinal, or colonic tissue type, or any combination thereof.
25. The method of alternative 24, wherein the tissue type of the one or more organoids and the tissue type of the organoid or enteroid are different.
26. The method of alternative 24, wherein the tissue type of the one or more organoids and the tissue type of the organoid or enteroid are the same.
27. The method of alternative 26, wherein the one or more organoids and the organoid or enteroid each comprises only one of the esophageal, gastric, hepatic, intestinal, or colonic tissue type, such that the resultant composite organoid is a homogenous organoid comprising one tissue type.
28. The method of any preceding alternative, wherein the one or more organoids is derived from pluripotent stem cells (PSCs) from a first subject, and the organoid or enteroid is derived from PSCs or isolated from gastrointestinal tissue from a second subject.
29. The method of alternative 28, wherein the first subject and the second subject are mammals.
30. The method of alternative 28 or 29, wherein the first subject and the second subject are humans.
31. The method of any one of alternatives 28-30, wherein the first subject and the second subject are the same individual.
32. The method of any one of the preceding alternatives, further comprising transplanting the composite organoid to a recipient subject.
33. The method of alternative 32, wherein the recipient subject is the first subject or the second subject.
34. The method of alternative 32 or 33, wherein the composite organoid exhibits greater engraftment and growth in the recipient subject compared to a comparable non-composite organoid or enteroid.
35. A composite organoid, comprising
a mesenchyme comprising mono-dissociated mesenchymal cells isolated from a first organoid; and
an epithelium comprising an epithelial structure isolated from a second organoid or enteroid.
36. The organoid of alternative 35, wherein the ratio of the number of mono-dissociated mesenchymal cells to epithelial cells in the composite organoid is greater than that of the second organoid or enteroid.
37. The organoid of alternative 35 or 36, wherein the first organoid and second organoid or enteroid each comprises a tissue type selected from an esophageal, gastric, hepatic, intestinal, or colonic tissue type, or any combination thereof.
38. The organoid of alternative 37, wherein the tissue type of the first organoid and the tissue type of the second organoid or enteroid is the same.
39. The organoid of alternative 37, wherein the tissue type of the first organoid and the tissue type of the second organoid or enteroid is different.
40. The organoid produced by the method of any one of alternatives 20-34.
41. The organoid of any one of alternatives 35-40 for use in treating a gastrointestinal malady in a subject in need thereof.

Additional embodiments of the present disclosure provided herein are described by way of the following additional numbered alternatives:

A method of producing a composite organoid, comprising:
a) obtaining mono-dissociated mesenchymal cells isolated from one or more organoids;
b) obtaining an epithelial structure isolated from an organoid or enteroid;
c) combining the mono-dissociated mesenchymal cells and the epithelial structure; and
d) culturing the combined mono-dissociated mesenchymal cells and the epithelial structure to form the composite organoid.

The method of alternative 1, wherein obtaining the mono-dissociated mesenchymal cells comprises isolating the mono-dissociated mesenchymal cells from the one or more organoids.

The method of alternative 1 or 2, wherein obtaining the epithelial structure comprises isolating the epithelial structure from the organoid or enteroid.

The method of any one of alternatives 1-3, wherein the mono-dissociated mesenchymal cells, or the epithelial structure, or both, are isolated by mechanical dissociation and filtration.

The method of any one of the preceding alternatives, wherein the mono-dissociated mesenchymal cells and the epithelial structure are combined by centrifugation.

The method of any one of the preceding alternatives, wherein the number of mesenchymal cells in the composite organoid is greater than the original number of mesenchymal cells of the organoid or enteroid from which the epithelial structure is isolated, such that the composite organoid has an enriched mesenchyme.

The method of any one of the preceding alternatives, wherein 1) the one or more organoids from which the mono-dissociated mesenchymal cells are isolated and 2) the organoid or enteroid from which the epithelial structure is isolated each comprises a tissue type selected from an esophageal, gastric, hepatic, intestinal, or colonic tissue type, or any combination thereof.

The method of any one of the preceding alternatives, wherein 1) the one or more organoids from which the mono-dissociated mesenchymal cells are isolated comprise an intestinal tissue type, and 2) the organoid or enteroid from which the epithelial structure is isolated comprises a tissue type selected from an esophageal, gastric, hepatic, intestinal, or colonic tissue type, or any combination thereof.

The method of any one of the preceding alternatives, wherein the one or more organoids from which the mono-dissociated mesenchymal cells are isolated are small intestinal organoids, optionally human intestinal organoids (HIOs).

The method of any one of alternatives 7-9, wherein the tissue type of the one or more organoids from which the mono-dissociated mesenchymal cells are isolated and the tissue type of the organoid or enteroid from which the epithelial structure is isolated are different.

The method of alternative 10, wherein the tissue type of the one or more organoids from which the mono-dissociated mesenchymal cells are isolated and the tissue type of the organoid or enteroid from which the epithelial structure is isolated are different, wherein no repatterning of the epithelial structure by the mono-dissociated mesenchymal cells occurs, such that the epithelium of the composite organoid maintains the tissue type of the organoid or enteroid,
optionally wherein the organoid or enteroid from which the epithelial structure is isolated is an organoid, and the organoid from which the epithelial structure is isolated is at least 14, 15, 16, 17, 18, 19, 20, 21, 22, 23, 24, 25, 26, 27, 28, 29, or 30 days old, or between 14-30, 15-30, 18-30, 15-20, or 15-25 days old;
or optionally wherein the organoid or enteroid from which the epithelial structure is isolated is an enteroid, wherein the enteroid is derived from adult tissue.

The method of alternative 10, wherein the organoid or enteroid from which the epithelial structure is isolated is an organoid, wherein the tissue type of the one or more organoids from which the mono-dissociated mesenchymal cells and the tissue type of the organoid from which the epithelial structure is isolated are different, and wherein repatterning of the epithelial structure by the mono-dissociated mesenchymal cells occurs, such that the epithelium of the composite organoid exhibits properties of the tissue type of the one or more organoids, optionally wherein the organoid from which the epithelial structure is isolated is no more than 8, 9, 10, 11, 12, or 13 days old or between 8-13, 8-10, or 10-13 days old.

The method of any one of alternatives 7-9, wherein the tissue type of the one or more organoids from which the mono-dissociated mesenchymal cells are isolated and the tissue type of the organoid or enteroid from which the epithelial structure is isolated are the same.

The method of alternative 13, wherein the one or more organoids and the organoid or enteroid each comprises only one of the esophageal, gastric, hepatic, intestinal, or colonic tissue type, such that the resultant composite organoid is a homogenous organoid comprising one tissue type.

The method of any preceding alternative, wherein the one or more organoids is derived from pluripotent stem cells (PSCs) from a first subject, and the organoid or enteroid is derived from PSCs or isolated from gastrointestinal tissue from a second subject.

The method of alternative 15, wherein the first subject and the second subject are mammals.

The method of alternative 15 or 16, wherein the first subject and the second subject are humans.

The method of any one of alternatives 15-17, wherein the first subject and the second subject are the same individual.

The method of any one of the preceding alternatives, further comprising transplanting the composite organoid to a recipient subject.

The method of alternative 19, wherein the recipient subject is the first subject or the second subject.

The method of alternative 19 or 20, wherein the composite organoid exhibits greater engraftment and growth in the recipient subject compared to a comparable non-composite organoid or enteroid.

The method of any one of the preceding alternatives, wherein the 1) the one or more organoids from which the mono-dissociated mesenchymal cells are isolated or 2) the organoid or enteroid from which the epithelial structure is isolated, or both, are engineered to comprise one or more exogenous nucleic acids or proteins.

The method of any one of the preceding alternatives, wherein the 1) the one or more organoids from which the mono-dissociated mesenchymal cells are isolated or 2) the organoid or enteroid from which the epithelial structure is isolated, or both, comprise a genetic mutation, optionally wherein the genetic mutation is associated with a disease state or a model of a disease state.

A composite organoid, comprising:
a mesenchyme comprising mesenchymal cells isolated as mono-dissociated cells from a first organoid; and
an epithelium comprising epithelial cells isolated as an epithelial structure from a second organoid or enteroid.

The composite organoid of alternative 24, wherein the ratio of the number of mesenchymal cells to epithelial cells in the composite organoid is greater than that of the second organoid or enteroid.

The composite organoid of alternative 24 or 25, wherein the first organoid and second organoid or enteroid each comprises a tissue type selected from an esophageal, gastric, hepatic, intestinal, or colonic tissue type, or any combination thereof.

The composite organoid of alternative 26, wherein the tissue type of the first organoid and the tissue type of the second organoid or enteroid is the same.

The composite organoid of alternative 26, wherein the tissue type of the first organoid and the tissue type of the second organoid or enteroid is different.

The composite organoid of alternative 28, wherein the tissue type of the first organoid from which the mesenchymal cells are isolated and the tissue type of the second organoid or enteroid from which the epithelial cells are isolated is different, wherein no repatterning of the epithelial cells by the mesenchymal cells occurs, such that the epithelium of the composite organoid maintains the tissue type of the second organoid or enteroid from which the epithelial cells are isolated,
optionally wherein the second organoid or enteroid is a second organoid, and the second organoid is at least 14, 15, 16, 17, 18, 19, 20, 21, 22, 23, 24, 25, 26, 27, 28, 29, or 30 days old or between 14-30, 15-30, 18-30, 15-20, or 15-25 days old; or
optionally wherein the second organoid or enteroid is a second enteroid, wherein the second enteroid is derived from adult tissue.

The composite organoid of alternative 28, wherein the second organoid or enteroid is a second organoid, wherein the tissue type of the first organoid from which the mesenchymal cells are isolated and the tissue type of the second organoid from which the epithelial cells are isolated is different, and wherein repatterning of the epithelial cells by the mesenchymal cells occurs, such that the epithelium of the composite organoid exhibits properties of the tissue type of the first organoid from which the mesenchymal cells are isolated, optionally wherein the second organoid is no more than 8, 9, 10, 11, 12, or 13 days old or between 8-13, 8-10, or 10-13 days old.

A composite organoid, comprising:
a mesenchyme comprising a first tissue type selected from an esophageal, gastric, hepatic, intestinal, or colonic tissue type, or any combination thereof, and
an epithelium comprising a second tissue type selected from an esophageal, gastric, hepatic, intestinal, or colonic tissue type, or any combination thereof,
wherein the first tissue type and second tissue type have at least one difference in tissue types.

The composite organoid of any one of alternatives 24-31, comprising one or more exogenous nucleic acids or proteins.

The composite organoid of any one of alternatives 24-32, wherein the composite organoid has or is engineered to comprise a genetic mutation, optionally wherein the genetic mutation is associated with a disease state or a model of a disease state.

The organoid produced by the method of any one of alternatives 1-23.

The organoid of any one of alternatives 24-34 for use in treating a gastrointestinal malady in a subject in need thereof.

A method for screening for a candidate therapeutic, comprising contacting the organoid of any one of alternatives 24-34 with the candidate therapeutic and determining the effect of the candidate therapeutic on the organoid.

The method of alternative 36, wherein the organoid is genetically modified, optionally genetically modified to exhibit a disease or model thereof.

The method of alternative 36 or 37, wherein the mesenchyme and/or the epithelium of the organoid is genetically modified, optionally genetically modified to exhibit a disease or model thereof.

### BRIEF DESCRIPTION OF THE DRAWINGS

In addition to the features described herein, additional features and variations will be readily apparent from the following descriptions of the drawings and exemplary embodiments. It is to be understood that these drawings depict embodiments and are not intended to be limiting in scope.

FIG. 1 depicts an embodiment of representative pictures of key steps of the dissociation/recombination protocol. Representative pictures of in vitro organoids before (top) and after (bottom) manual dissociation from day 14 HIOs (panel A). Image of a 96 well plate well containing mono-dissociated mesenchyme and one epithelial structure after centrifugation (panel B). Image of a composite organoid 24 hours after recombination before plating in Matrigel (panel C).

FIG. 2A depicts an embodiment of the formation of HIO-mesenchyme/HCO-epithelium heterogeneous organoids. Images show the organization of the heterogeneous organoid 48 hours and 9 days after recombination. The HCO epithelium is positive for GFP fluorescence.

FIG. 2B depicts an embodiment of the engraftment of a HIO-mesenchyme/HCO-epithelium heterogeneous organoid to kidney capsule tissue of a mouse model. The heterogeneous organoid exhibits robust maturation and engraftment comparable to HIO organoids and greater than HCO organoids.

FIG. 2C depicts an embodiment of immunofluorescence images showing that the HIO-mesenchyme/HCO-epithelium heterogeneous organoid derived from day 18 source organoids is positive for large intestine-specific special AT-rich sequence binding protein 2 (SATB2) and negative for small intestine-specific sucrase-isomaltase (SI), indicating that epithelial structures derived from HCOs at this age maintain their large intestine identity.

FIG. 2D depicts an embodiment of immunofluorescence images showing that the HIO-mesenchyme/HCO-epithelium heterogeneous organoid derived from day 11 source organoids (compared to day 18 source organoids) is positive for small intestine-specific GATA binding protein 4 (GATA4) and negative for colon-specific SATB2, indicating that epithelial structures derived from HCOs at this age can be reprogrammed by the surrounding mesenchyme (e.g., to change large intestine epithelium into a small intestine identity).

FIG. 2E depicts an embodiment of the formation of HIO-mesenchyme/HGO-epithelium heterogeneous organoids. Images show the organization of the heterogeneous organoid 4 days and 11 days after recombination. Immunofluorescence staining confirms maturation of GFP-positive intestinal mesenchyme and CDH1-positive gastric epithelium.

FIG. 2F depicts an embodiment of the formation of HIO-mesenchyme/enteroid organoids. Images show the organization of the heterogeneous organoid 10 days, 21 days, and 31 days after recombination. The enteroids is positive for GFP fluorescence.

FIG. 3 depicts an embodiment of images depicting the formation of heterogeneous organoids of different types and engraftment to the kidney capsule of a mouse model. HIO-mesenchyme/HIO-epithelium (panel A), HIO-mesenchyme/HCO-epithelium (panel B), and HIO-mesenchyme/HAGO-epithelium (panel C) heterogeneous organoids were tested. Also formed are HIO-mesenchyme-enteroid (panel D), HAGO-mesenchyme/HAGO-epithelium (panel E), and HCO-mesenchyme/HCO-epithelium (panel F) organoids. The HAGO and HCO homogeneous organoids were prepared by enriching HAGO or HCO mesenchymal cells from a large number of source organoids and recombining these mesenchymal cells with an epithelial structure.

FIG. 4A depicts an embodiment of images taken 48 hours after recombination between GFP-positive HIO mesenchyme and GFP-negative HEO epithelium in vitro.

FIG. 4B depicts an embodiment of images of recombined HIO/HEO grafts 8 weeks after transplantation under the kidney capsule of NSG mice.

FIG. 4C depicts an embodiment of H&E staining of a transplanted HIO/HEO tissue.

FIG. 4D depicts an embodiment of GFP and CDH1 staining showing the purity of the recombination after engraftment.

FIG. 4E depicts an embodiment of immunostaining of transplanted HIO/HEO tissue revealing the development and maturity of the esophageal epithelium, including basal layers (KRT5 and KRT14), supra-basal layers (KRT13 and IVL), and positivity for the esophageal-specific transcription factor p63.

### DETAILED DESCRIPTION

Recent methods to pattern foregut (e.g. human gastric organoids [HGO]) and hindgut (e.g. human colonic organoids [HCO]) in vitro result in heterogenicity in epithelial to mesenchymal ratios that prevent or limit the ability of these structures to engraft in vivo. Herein, Applicant shows that the lack of a robust mesenchyme reduces the rate of successful engraftment in animal models, and disclose methods and compositions with improved mesenchyme to address one or more of the limitations of prior organoids.

Disclosed herein are composite organoids and compositions thereof and methods of making the same involving the dissociation and re-association (recombination) of epithelial and mesenchymal components to form organoids having improved epithelial and mesenchymal ratios and morphologies. These organoids can be used for purposes such as drug screening or personalized medicine and are suitable for transplantation, for example, autologously or allogeneically to a subject, such as a human or other mammal, or xenogeneically into immunocompromised animals. In some embodiments, the composite organoids are composite gastrointestinal organoids. In other embodiments, the composite organoids are composite organoids of other tissue types, for example, brain, neuronal, muscle, thyroid, cardiac, pulmonary, kidney, or pancreatic organoids. In some embodiments, the epithelial and mesenchymal components are derived from donor organoids derived from pluripotent stem cells (PSCs) using a stepwise approach that mimics embryonic gut development, or from enteroids derived from PSCs or isolated from gastrointestinal tissue from a donor subject. In some embodiments, the organoids have tridimensional structures comprising a polarized epithelium surrounded by supporting mesenchymal cells. In some embodiments, the composite organoids or donor organoids, or both, comprise intestinal organoids, colonic organoids, gastric organoids, antral gastric organoids, fundic gastric organoids, hepatic organoids, or pancreatic organoids, or any combination thereof. In some embodiments, the epithelial components are derived from patient-derived enteroids. In some embodiments, the composite organoids or donor organoids, or both, are human. Methods of producing organoids or enteroids can be found in U.S. Pat. Nos. 9,719,068 and 10,174,289, and PCT Publications WO 2016/061464, WO 2017/192997, WO 2018/106628, WO 2018/200481, WO 2018/085615, WO 2018/085622, WO 2018/085623, WO 2018/226267, WO 2019/074793, WO 2020/023245, each of which is hereby expressly incorporated by reference in its entirety.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

### Terms

Unless defined otherwise, technical and scientific terms used herein have the same meaning as commonly understood when read in light of the instant disclosure by one of ordinary skill in the art to which the present disclosure belongs. For purposes of the present disclosure, the following terms are explained below.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 10% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

The terms “effective amount” or “effective dose” as used herein have their plain and ordinary meaning as understood in light of the specification, and refer to that amount of a recited composition or compound that results in an observable effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The terms “function” and “functional” as used herein have their plain and ordinary meaning as understood in light of the specification, and refer to a biological, enzymatic, or therapeutic function.

The term “inhibit” as used herein has its plain and ordinary meaning as understood in light of the specification, and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. As used herein, the term “delay” has its plain and ordinary meaning as understood in light of the specification, and refers to a slowing, postponement, or deferment of a biological event, to a time which is later than would otherwise be expected. The delay can be a delay of a percentage that is, is about, is at least, is at least about, is not more than, or is not more than about, 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning as understood in light of the specification and refers to the performance of a method inside living organisms, usually animals, mammals, including humans, and plants, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

The terms “nucleic acid” or “nucleic acid molecule” as used herein have their plain and ordinary meaning as understood in light of the specification, and refer to polynucleotides, such as deoxyribonucleic acid (DNA) or ribonucleic acid (RNA), oligonucleotides, those that appear in a cell naturally, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, retrovirus, lentivirus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein has its plain and ordinary meaning as understood in light of the specification and refers to a sequence being after the 3′-end of a previous sequence, on the strand containing the encoding sequence (sense strand) if the nucleic acid is double stranded. The term “upstream” on a nucleic acid as used herein has its plain and ordinary meaning as understood in light of the specification and refers to a sequence being before the 5′-end of a subsequent sequence, on the strand containing the encoding sequence (sense strand) if the nucleic acid is double stranded. The term “grouped” on a nucleic acid as used herein has its plain and ordinary meaning as understood in light of the specification and refers to two or more sequences that occur in proximity either directly or with extra nucleic acids in between, e.g. linkers, repeats, or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

Some embodiments described herein relate to pharmaceutical compositions that comprise, consist essentially of, or consist of an effective amount of a cell composition described herein and a pharmaceutically acceptable carrier, excipient, or combination thereof. A pharmaceutical composition described herein is suitable for human and/or veterinary applications.

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or “carrier” can refer to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, and nonionic surfactants such as TWEEN®, polyethylene glycol (PEG), and PLURONICS®. The composition, if desired, can also contain minor amounts of wetting, bulking, emulsifying agents, or pH buffering agents. These compositions can take the form of solutions, suspensions, emulsion, sustained release formulations and the like. The formulation typically suits the mode of administration.

Cryoprotectants are cell composition additives to improve efficiency and yield of low temperature cryopreservation by preventing formation of large ice crystals. Cryoprotectants include but are not limited to DMSO, ethylene glycol, glycerol, propylene glycol, trehalose, formamide, methyl-formamide, dimethyl-formamide, glycerol 3-phosphate, proline, sorbitol, diethyl glycol, sucrose, triethylene glycol, polyvinyl alcohol, polyethylene glycol, or hydroxyethyl starch. Cryoprotectants can be used as part of a cryopreservation medium, which include other components such as nutrients (e.g. albumin, serum, bovine serum, fetal calf serum [FCS]) to enhance post-thawing survivability of the cells. In these cryopreservation media, at least one cryoprotectant may be found at a concentration that is, is about, is at least, is at least about, is not more than, or is not more than about, 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90%, or any percentage within a range defined by any two of the aforementioned numbers.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in composition at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral delivery, including intramuscular, subcutaneous, intra-arterial, intravenous, intraportal, intra-articular, intradermal, peritoneal, intramedullary injections, intrathecal, direct intraventricular, intraperitoneal, intranasal or intraocular injections. Pharmaceutical compositions will generally be tailored to the specific intended route of administration.

As used herein, a “carrier” has its plain and ordinary meaning as understood in light of the specification and refers to a compound, particle, solid, semi-solid, liquid, or diluent that facilitates the passage, delivery and/or incorporation of a compound to cells, tissues and/or bodily organs.

As used herein, a “diluent” has its plain and ordinary meaning as understood in light of the specification and refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The term “purity” of any given substance, compound, or material as used herein has its plain and ordinary meaning as understood in light of the specification and refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to nucleic acids, DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the substance, compound, or material is substantially free of host cell proteins, host cell nucleic acids, plasmid DNA, contaminating viruses, proteasomes, host cell culture components, process related components, mycoplasma, pyrogens, bacterial endotoxins, and adventitious agents. Purity can be measured using technologies including but not limited to electrophoresis, SDS-PAGE, capillary electrophoresis, PCR, rtPCR, qPCR, chromatography, liquid chromatography, gas chromatography, thin layer chromatography, enzyme-linked immunosorbent assay (ELISA), spectroscopy, UV-visible spectrometry, infrared spectrometry, mass spectrometry, nuclear magnetic resonance, gravimetry, or titration, or any combination thereof.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

The term “mono-dissociated” as used herein has its plain and ordinary meaning as understood in light of the specification and refers to preparations of suspensions of single cells. These suspensions of single cells may be prepared by processing a multicellular tissue or structure, for example, through conventional mechanical and/or chemical (e.g. enzymatic) means. As used for the methods and compositions disclosed herein, mono-dissociated may apply to mesenchymal cells processed from multicellular tissues or structure such as organoids into suspensions of single cells. It should be understood that while “mono-dissociated” refers to populations of single cells, a preparation of mono-dissociated cells does not necessarily need to contain exclusively single cells, and preparations of mono-dissociated cells may comprise a population ofundissociated cell structures, cell clumps, aggregated cells, cell debris, and the like. Similarly, in some embodiments, compositions of mono-dissociated cells of a particular type (e.g. mesenchymal) may contain cells of another type (e.g. epithelial).

The term “epithelial structure” as used herein has its plain and ordinary meaning as understood in light of the specification and refers to multicellular tissues or fragments thereof made up of intact epithelial cells, for example, from a differentiated organoid or enteroid (although other sources of epithelial cells are envisioned). As used in the methods disclosed herein, these epithelial structures are cohesive clumps of epithelial cells that can be isolated, for example, using an appropriate cell strainer that will allow single cells to pass through but retain these larger epithelial structures.

The term “composite organoid” as used herein has its plain and ordinary meaning as understood in light of the specification and refers to cellular organoids comprising both a mesenchyme and epithelium, where the mesenchyme and epithelium are combined as described herein to form the composite organoid. As envisioned herein, these two cell populations may be derived from the same type of organoid or organoid having the same tissue type (for example, where the mesenchyme and epithelium are both derived from gastric organoids, but the gastric organoids used to isolate mesenchyme and at least some of the gastric organoids used to isolate epithelium are not the same organoids) or may be derived from different types of organoids or organoids having different tissue types (for example, where the mesenchyme is derived from intestinal organoids and the epithelium is derived from gastric organoids). In some embodiments, the organoids from which the mesenchyme and epithelium are derived may have some differences other than tissue type, even if the tissue type is the same, such as genetic modifications or exhibiting a disease phenotype. The combination of mesenchyme and epithelium to form a composite organoid is distinct from an organoid produced from pluripotent stem cells as previously disclosed, as the mesenchyme and epithelium of these organoids arise in tandem through a process of cell differentiation, for example, from pluripotent stem cell to definitive endoderm to gut endoderm for gastrointestinal organoids.

The term “% w/w” or “% wt/wt” as used herein has its plain and ordinary meaning as understood in light of the specification and refers to a percentage expressed in terms of the weight of the ingredient or agent over the total weight of the composition multiplied by 100. The term “% v/v” or “% vol/vol” as used herein has its plain and ordinary meaning as understood in the light of the specification and refers to a percentage expressed in terms of the liquid volume of the compound, substance, ingredient, or agent over the total liquid volume of the composition multiplied by 100.

The disclosure herein generally uses affirmative language to describe the numerous embodiments. The disclosure also includes embodiments in which subject matter is excluded, in full or in part, such as substances or materials, method steps and conditions, protocols, or procedures.

### Stem Cells

The term “totipotent stem cells” (also known as omnipotent stem cells) as used herein has its plain and ordinary meaning as understood in light of the specification and are stem cells that can differentiate into embryonic and extra-embryonic cell types. Such cells can construct a complete, viable organism. These cells are produced from the fusion of an egg and sperm cell. Cells produced by the first few divisions of the fertilized egg are also totipotent.

The term “embryonic stem cells (ESCs),” also commonly abbreviated as ES cells, as used herein has its plain and ordinary meaning as understood in light of the specification and refers to cells that are pluripotent and derived from the inner cell mass of the blastocyst, an early-stage embryo. For purpose of the present disclosure, the term “ESCs” is used broadly sometimes to encompass the embryonic germ cells as well.

The term “pluripotent stem cells (PSCs)” as used herein has its plain and ordinary meaning as understood in light of the specification and encompasses any cells that can differentiate into nearly all cell types of the body, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of inner cell mass cells of the preimplantation blastocyst or obtained through induction of a non-pluripotent cell, such as an adult somatic cell, by forcing the expression of certain genes. Pluripotent stem cells can be derived from any suitable source. Examples of sources of pluripotent stem cells include mammalian sources, including human, rodent, porcine, and bovine.

The term “induced pluripotent stem cells (iPSCs),” also commonly abbreviated as iPS cells, as used herein has its plain and ordinary meaning as understood in light of the specification and refers to a type of pluripotent stem cells artificially derived from a normally non-pluripotent cell, such as an adult somatic cell, by inducing a “forced” expression of certain genes. hiPSC refers to human iPSCs. In some methods known in the art, iPSCs may be derived by transfection of certain stem cell-associated genes into non-pluripotent cells, such as adult fibroblasts. Transfection may be achieved through viral transduction using viruses such as retroviruses or lentiviruses. Transfected genes may include the master transcriptional regulators Oct-3/4 (POU5F1) and Sox2, although other genes may enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include first generation iPSCs, second generation iPSCs in mice, and human induced pluripotent stem cells. In some methods, a retroviral system is used to transform human fibroblasts into pluripotent stem cells using four pivotal genes: Oct3/4, Sox2, Klf4, and c-Myc. In other methods, a lentiviral system is used to transform somatic cells with OCT4, SOX2, NANOG, and LIN28. Genes whose expression are induced in iPSCs include but are not limited to Oct-3/4 (POU5F1); certain members of the Sox gene family (e.g., Sox1, Sox2, Sox3, and Sox15); certain members of the Klf family (e.g., Klf1, Klf2, Klf4, and Klf5), certain members of the Myc family (e.g., C-myc, L-myc, and N-myc), Nanog, LIN28, Tert, Fbx15, ERas, ECAT15-1, ECAT15-2, Tcl1, β-Catenin, ECAT1, Esg1, Dnmt3L, ECAT8, Gdf3, Fth117, Sal14, Rex1, UTF1, Stella, Stat3, Grb2, Prdm14, Nr5a1, Nr5a2, or E-cadherin, or any combination thereof.

The term “precursor cell” as used herein has its plain and ordinary meaning as understood in light of the specification and encompasses any cells that can be used in methods described herein, through which one or more precursor cells acquire the ability to renew itself or differentiate into one or more specialized cell types. In some embodiments, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment. Precursor cells include embryonic stem cells (ESC), embryonic carcinoma cells (ECs), and epiblast stem cells (EpiSC).

In some embodiments, one step is to obtain stem cells that are pluripotent or can be induced to become pluripotent. In some embodiments, pluripotent stem cells are derived from embryonic stem cells, which are in turn derived from totipotent cells of the early mammalian embryo and are capable of unlimited, undifferentiated proliferation in vitro. Embryonic stem cells are pluripotent stem cells derived from the inner cell mass of the blastocyst, an early-stage embryo. Methods for deriving embryonic stem cells from blastocytes are well known in the art. Human embryonic stem cells (e.g. H1, H7, or H9 ESC lines) are used in the exemplary embodiments described in the present application, but it would be understood by one of skill in the art that the methods and systems described herein are applicable to any stem cells.

Additional stem cells that can be used in embodiments in accordance with the present disclosure include but are not limited to those provided by or described in the database hosted by the National Stem Cell Bank (NSCB), Human Embryonic Stem Cell Research Center at the University of California, San Francisco (UCSF); WISC cell Bank at the Wi Cell Research Institute; the University of Wisconsin Stem Cell and Regenerative Medicine Center (UW-SCRMC); Novocell, Inc. (San Diego, Calif.); Cellartis AB (Goteborg, Sweden); ES Cell International Pte Ltd (Singapore); Technion at the Israel Institute of Technology (Haifa, Israel); and the Stem Cell Database hosted by Princeton University and the University of Pennsylvania. Exemplary embryonic stem cells that can be used in embodiments in accordance with the present disclosure include but are not limited to SA01 (SA001); SA02 (SA002); ES01 (HES-1); ES02 (HES-2); ES03 (HES-3); ES04 (HES-4); ES05 (HES-5); ES06 (HES-6); BG01 (BGN-01); BG02 (BGN-02); BG03 (BGN-03); TE03 (13); TE04 (14); TE06 (16); UC01 (HSF1); UC06 (HSF6); WA01 (H1); WA07 (H7); WA09 (H9); WA13 (H13); WA14 (H14). Exemplary human pluripotent cell lines include but are not limited to TkDA3-4, 1231A3, 317-D6, 317-A4, CDH1, 5-T-3, 3-34-1, NAFLD27, NAFLD77, NAFLD150, WD90, WD91, WD92, L20012, C213, 1383D6, FF, or 317-12 cells.

In developmental biology, cellular differentiation is the process by which a less specialized cell becomes a more specialized cell type. As used herein, the term “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

In some embodiments, an adenovirus can be used to transport the requisite four genes, resulting in iPSCs substantially identical to embryonic stem cells. Since the adenovirus does not combine any of its own genes with the targeted host, the danger of creating tumors is eliminated. In some embodiments, non-viral based technologies are employed to generate iPSCs. In some embodiments, reprogramming can be accomplished via plasmid without any virus transfection system at all, although at very low efficiencies. In other embodiments, direct delivery of proteins is used to generate iPSCs, thus eliminating the need for viruses or genetic modification. In some embodiment, generation of mouse iPSCs is possible using a similar methodology: a repeated treatment of the cells with certain proteins channeled into the cells via poly-arginine anchors was sufficient to induce pluripotency. In some embodiments, the expression of pluripotency induction genes can also be increased by treating somatic cells with FGF2 under low oxygen conditions.

The term “feeder cell” as used herein has its plain and ordinary meaning as understood in light of the specification and refers to cells that support the growth of pluripotent stem cells, such as by secreting growth factors into the medium or displaying on the cell surface. Feeder cells are generally adherent cells and may be growth arrested. For example, feeder cells are growth-arrested by irradiation (e.g. gamma rays), mitomycin-C treatment, electric pulses, or mild chemical fixation (e.g. with formaldehyde or glutaraldehyde). However, feeder cells do not necessarily have to be growth arrested. Feeder cells may serve purposes such as secreting growth factors, displaying growth factors on the cell surface, detoxifying the culture medium, or synthesizing extracellular matrix proteins. In some embodiments, the feeder cells are allogeneic or xenogeneic to the supported target stem cell, which may have implications in downstream applications. In some embodiments, the feeder cells are mouse cells. In some embodiments, the feeder cells are human cells. In some embodiments, the feeder cells are mouse fibroblasts, mouse embryonic fibroblasts, mouse STO cells, mouse 3T3 cells, mouse SNL 76/7 cells, human fibroblasts, human foreskin fibroblasts, human dermal fibroblasts, human adipose mesenchymal cells, human bone marrow mesenchymal cells, human amniotic mesenchymal cells, human amniotic epithelial cells, human umbilical cord mesenchymal cells, human fetal muscle cells, human fetal fibroblasts, or human adult fallopian tube epithelial cells. In some embodiments, conditioned medium prepared from feeder cells is used in lieu of feeder cell co-culture or in combination with feeder cell co-culture. In some embodiments, feeder cells are not used during the proliferation of the target stem cells.

### Differentiation of PSCs

In some embodiments, PSCs, such as ESCs and iPSCs, undergo directed differentiation in a stepwise manner first into definitive endoderm (DE) cells, then into foregut or hindgut lineages, and into tissue such as gastrointestinal tissue or any other biological tissue. In some embodiments, PSCs undergo directed differentiation in a non-stepwise manner where molecules (e.g. growth factors, ligands) for promoting DE formation and for subsequent tissue formation are added at the same time. In some embodiments, directed differentiation is achieved by selectively activating certain signaling pathways in the iPSCs and/or DE cells. In some embodiments, the signaling pathways include but not limited to the Wnt signaling pathway; Wnt/APC signaling pathway; FGF signaling pathway; TGF-beta signaling pathway; BMP signaling pathway; Notch signaling pathway; Hedgehog signaling pathway; LKB signaling pathway; and Par polarity signaling pathway.

The definitive endoderm gives rise to the gut tube. The anterior DE forms the foregut and its associated organs including the esophagus, lungs, stomach, liver and pancreas and the posterior DE forms the midgut and hindgut, which forms the small and large intestines and parts of the genitourinary system. Studies using mouse, chick and frog embryos suggest that establishing the anterior-posterior pattern in DE at the gastrula stage is a prerequisite for subsequent foregut and hindgut development. The Wnt and FGF signaling pathways are critical for promoting either posterior endoderm/hindgut or anterior endoderm/foregut fate. In hindgut, the simple cuboidal epithelium first develops into a pseudostratified columnar epithelium, then into villi containing a polarized columnar epithelium and a proliferative zone at the base of the villi, which corresponds with the presumptive progenitor domain.

Any applicable methods for producing definitive endoderm from pluripotent cells (e.g., iPSCs or ESCs) can be used in the methods described herein. In some embodiments, pluripotent cells are derived from a morula. In some embodiments, pluripotent stem cells are stem cells. Stem cells used in these methods can include, but are not limited to, embryonic stem cells. Embryonic stem cells can be derived from the embryonic inner cell mass or from the embryonic gonadal ridges. Embryonic stem cells or germ cells can originate from a variety of animal species including, but not limited to, various mammalian species including humans. In some embodiments, human embryonic stem cells are used to produce definitive endoderm. In some embodiments, human embryonic germ cells are used to produce definitive endoderm. In some embodiments, iPSCs are used to produce definitive endoderm. In some embodiments, human iPSCs (hiPSCs) are used to produce definitive endoderm. In some embodiments, PSCs are first modified before differentiating into definitive endoderm. In some embodiments, the PSCs are genetically modified, such as to express an exogenous nucleic acid or protein, before differentiating into definitive endoderm.

In some embodiments, the embryonic stem cells or germ cells or iPSCs are treated with one or more small molecule compounds, activators, inhibitors, or growth factors for a time that is, is about, is at least, is at least about, is not more than, or is not more than about, 6 hours, 12 hours, 18 hours, 24 hours, 36 hours, 48 hours, 60 hours, 72 hours, 84 hours, 96 hours, 120 hours, 150 hours, 180 hours, 240 hours, 300 hours or any time within a range defined by any two of the aforementioned times, for example 6 hours to 300 hours, 24 hours to 120 hours, 48 hours to 96 hours, 6 hours to 72 hours, or 24 hours to 300 hours. In some embodiments, more than one small molecule compounds, activators, inhibitors, or growth factors are added. In these cases, the more than one small molecule compounds, activators, inhibitors, or growth factors can be added simultaneously or separately.

In some embodiments, the embryonic stem cells or germ cells or iPSCs are treated with one or more small molecule compounds, activators, inhibitors, or growth factors at a concentration that is, is about, is at least, is at least about, is not more than, or is not more than about, 10 ng/mL, 20 ng/mL, 50 ng/mL, 75 ng/mL, 100 ng/mL, 120 ng/mL, 150 ng/mL, 200 ng/mL, 500 ng/mL, 1000 ng/mL, 1200 ng/mL, 1500 ng/mL, 2000 ng/mL, 5000 ng/mL, 7000 ng/mL, 10000 ng/mL, or 15000 ng/mL, or any concentration that is within a range defined by any two of the aforementioned concentrations, for example, 10 ng/mL to 15000 ng/mL, 100 ng/mL to 5000 ng/mL, 500 ng/mL to 2000 ng/mL, 10 ng/mL to 2000 ng/mL, or 1000 ng/mL to 15000 ng/mL. In some embodiments, concentration of the one or more small molecule compounds, activators, inhibitors, or growth factors is maintained at a constant level throughout the treatment. In some embodiments, concentration of the one or more small molecule compounds, activators, inhibitors, or growth factors is varied during the course of the treatment. In some embodiments, more than one small molecule compounds, activators, inhibitors, or growth factors are added. In these cases, the more than one small molecule compounds, activators, inhibitors, or growth factors can differ in concentrations.

In some embodiments, the ESCs, germ cells, or iPSCs are cultured in growth media that supports the growth of stem cells. In some embodiments, the ESCs, germ cells, or iPSCs are cultured in stem cell growth media. In some embodiments, the stem cell growth media is RPMI 1640, DMEM, DMEM/F12, Minigut media, mTeSR 1, or mTeSR Plus media. In some embodiments, the stem cell growth media comprises fetal bovine serum (FBS). In some embodiments, the stem cell growth media comprises FBS at a concentration that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or 20%, or any percentage within a range defined by any two of the aforementioned concentrations, for example 0% to 20%, 0.2% to 10%, 2% to 5%, 0% to 5%, or 2% to 20%. In some embodiments, the stem cell growth media does not contain xenogeneic components. In some embodiments, the growth media comprises one or more small molecule compounds, activators, inhibitors, or growth factors.

In some embodiments, populations of cells enriched in definitive endoderm cells are used. In some embodiments, the definitive endoderm cells are isolated or substantially purified. In some embodiments, the isolated or substantially purified definitive endoderm cells express one or more (e.g. at least 1, 3) of SOX17, FOXA2, or CXRC4 markers to a greater extent than one or more (e.g. at least 1, 3, 5) of OCT4, AFP, TM, SPARC, or SOX7 markers.

In some embodiments, definitive endoderm cells and hESCs are treated with one or more growth factors. Such growth factors can include growth factors from the TGF-beta superfamily. In some embodiments, the one or more growth factors comprise the Nodal/Activin and/or the BMP subgroups of the TGF-beta superfamily of growth factors. In some embodiments, the one or more growth factors are selected from the group consisting of Nodal, Activin A, Activin B, BMP4, a Wnt protein or combinations of any of these growth factors. For example, in human, Wnt proteins include but are not limited to Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and Wnt16.

In some embodiments, activin-induced definitive endoderm (DE) can further undergo FGF/Wnt induced anterior or posterior endoderm pattering, foregut or hindgut specification and morphogenesis, and finally gastrointestinal growth, morphogenesis and cytodifferentiation into functional gastrointestinal cell types. In some embodiments, human PSCs are efficiently directed to differentiate in vitro into gastrointestinal epithelium or mesenchyme that includes secretory, endocrine and absorptive cell types. It will be understood that molecules such as growth factors can be added to any stage of the development to promote a particular type of gastrointestinal tissue formation.

Human gastrointestinal development in vitro occurs in stages that approximate fetal gut development; endoderm formation, anterior or posterior endoderm patterning, foregut or hindgut morphogenesis, fetal gut development, epithelial morphogenesis, formation of a presumptive progenitor domain, and differentiation into functional cell types.

It will be understood by one of skill in the art that altering the concentration, expression or function of one or more Wnt signaling proteins in combination with altering the concentration, expression, or function of one or more FGF proteins can give rise to directed differentiation in accordance of the present disclosure. In some embodiments, cellular constituents associated with the Wnt and/or FGF signaling pathways, for example, natural inhibitors, antagonists, activators, or agonists of the pathways can be used to result in inhibition or activation of the Wnt and/or FGF signaling pathways. In some embodiments, siRNA and/or shRNA targeting cellular constituents associated with the Wnt and/or FGF signaling pathways are used to inhibit or activate these pathways.

Fibroblast growth factors (FGFs) are a family of growth factors involved in angiogenesis, wound healing, and embryonic development. The FGFs are heparin-binding proteins and interactions with cell-surface associated heparan sulfate proteoglycans have been shown to be essential for FGF signal transduction. FGFs are key players in the processes of proliferation and differentiation of wide variety of cells and tissues. In humans, 22 members of the FGF family have been identified, all of which are structurally related signaling molecules. Members FGF1 through FGF10 all bind fibroblast growth factor receptors (FGFRs). FGF1 is also known as acidic fibroblast growth factor, and FGF2 is also known as basic fibroblast growth factor (bFGF). Members FGF11, FGF12, FGF13, and FGF14, also known as FGF homologous factors 1-4 (FHF1-FHF4), have been shown to have distinct functional differences compared to the FGFs. Although these factors possess remarkably similar sequence homology, they do not bind FGFRs and are involved in intracellular processes unrelated to the FGFs. This group is also known as “iFGF.” Members FGF15 through FGF23 are newer and not as well characterized. FGF15 is the mouse ortholog of human FGF19 (hence there is no human FGF15). Human FGF20 was identified based on its homology to Xenopus FGF-20 (XFGF-20). In contrast to the local activity of the other FGFs, FGF15/FGF19, FGF21 and FGF23 have more systemic effects.

In some embodiments, it will be understood by one of skill in the art that any of the FGFs can be used in conjunction with a protein from the Wnt signaling pathway. In some embodiments, the FGF used is one or more of FGF1, FGF2, FGF3, FGF4, FGF4, FGF5, FGF6, FGF7, FGF8, FGF8, FGF9, FGF10, FGF11, FGF12, FGF13, FGF14, FGF15 (FGF19, FGF15/FGF19), FGF16, FGF17, FGF18, FGF20, FGF21, FGF22, FGF23.

Differentiation of PSCs into DE culture and subsequently into various intermediate mature gastrointestinal cell types can be determined by the presence of stage-specific cell markers. In some embodiments, expression of representative cellular constituents is used to determine DE formation. The representative cellular constituents include but are not limited to CMKOR1, CXCR4, GPR37, RTN4RL1, SLC5A9, SLC40A1, TRPA1, AGPAT3, APOA2, C20orf56, C21orf129, CALCR, CCL2, CER1, CMKOR1, CRIP1, CXCR4, CXorf1, DIO3, DIO30S, EB-1, EHHADH, ELOVL2, EPSTI1, FGF17, FLJ10970, FLJ21195, FLJ22471, FLJ23514, FOXA2, FOXQ1, GATA4, GPR37, GSC, LOC283537, MYL7, NPPB, NTN4, PRSS2, RTN4RL1, SEMA3E, SIAT8D, SLC5A9, SLC40A1, SOX17, SPOCK3, TMOD1, TRPA1, TTN, AW166727, AI821586, BF941609, AI916532, BC034407, N63706 or AW772192, or any combination thereof. In some embodiments, the absence of cellular constituents, such as foregut markers Pdx1 and Albumin, can be used to reveal directed hindgut formation. In some embodiments, one or more (e.g. at least 1, 2, 3) intestinal transcription factors CDX2, KLF5 or SOX9 can be used to represent intestinal development. In some embodiments, one or more of GATA4 or GATA6 protein expression can be used to represent intestinal development.

In some embodiments, morphological changes can be used to represent the progress of directed differentiation. In some embodiments, spheroids (e.g., mid-hindgut, hindgut, anterior foregut, or posterior foregut spheroids) are subject to 3-dimensional culture conditions for maturation. In some embodiments, the gastrointestinal organoids mature in a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days, or any number of days within a range defined by any two of the aforementioned number of days, for example, 1 to 40 days, 20 to 30 days, 30 to 40 days, or 1 to 20 days. In some embodiments, a highly convoluted epithelium surrounded by mesenchymal cells can be observed following spheroid formation. In some embodiments, gastrointestinal organoids, epithelium, polarized columnar epithelium, mesenchyme, neuronal cells, or smooth muscle cells can be observed in a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days, or any number of days within a range defined by any two of the aforementioned number of days, for example, 1 to 40 days, 20 to 30 days, 30 to 40 days, or 1 to 20 days.

In some embodiments, pluripotent stem cells are converted into gastrointestinal cell types via a “one step” process. For example, one or more molecules that can differentiate pluripotent stem cells into DE culture (e.g., Activin A) are combined with additional molecules that can promote directed differentiation of DE culture (e.g., Wnt3a and FGF4) to directly treat pluripotent stem cells.

In some embodiments, pluripotent stem cells are prepared from somatic cells. In some embodiments, pluripotent stem cells are prepared from biological tissue obtained from a biopsy. In some embodiments, pluripotent stem cells are prepared from peripheral blood mononuclear cells (PBMCs). In some embodiments, human PSCs are prepared from human PBMCs. In some embodiments, pluripotent stem cells are prepared from cryopreserved PBMCs. In some embodiments, PBMCs are grown on a feeder cell substrate. In some embodiments, PBMCs are grown on a mouse embryonic fibroblast (MEF) feeder cell substrate. In some embodiments, PBMCs are grown on an irradiated MEF feeder cell substrate. In some embodiments, PBMCs are grown on 0.1% gelatin.

In some embodiments, pluripotent stem cells are prepared from PBMCs by viral transduction. In some embodiments, PBMCs are transduced with Sendai virus, lentivirus, adenovirus, or adeno-associated virus, or any combination thereof. In some embodiments, PBMCs are transduced with Sendai virus comprising expression vectors for Oct3/4, Sox2, Klf4, or L-Myc, or any combination thereof. In some embodiments, PBMCs are transduced with one or more viruses at an MOI that is, is about, is at least, is at least about, is not more than, or is not more than about, 0, 0.1, 0.5, 1.0, 1.5, 2.0, 2.5, 3.0, 3.5, 4.0, 4.5, or 5.0 MOI, or any MOI within a range defined by any two of the aforementioned MOIs, for example, 0 to 5.0, 1.0 to 4.0, 2.0 to 3.0, 0 to 3.0, or 1.0 to 5.0. In some embodiments, after transduction, PBMCs express stem cell reprogramming factors. In some embodiments, after transduction, PBMCs are reprogrammed to iPSCs. In some embodiments, iPSCs are grown on a feeder cell substrate. In some embodiments, iPSCs are grown on a MEF feeder cell substrate. In some embodiments, iPSCs are grown on an irradiated MEF feeder cell substrate. In some embodiments, iPSCs are grown on 0.1% gelatin. In some embodiments, iPSCs are grown in RPMI 1640, DMEM, DMEM/F12, Minigut media, mTeSR 1, or mTeSR Plus media.

In some embodiments, iPSCs are expanded in cell culture. In some embodiments, iPSCs are expanded in an extracellular matrix, or mimetic or derivative thereof. In some embodiments, iPSCs are expanded in Matrigel. In some embodiments, iPSCs are expanded in cell culture media comprising a ROCK inhibitor (e.g. Y-27632). In some embodiments, iPSCs are expanded until 80-95% confluence. In some embodiments, the iPSCs are differentiated into definitive endoderm cells. In some embodiments, iPSCs are differentiated into definitive endoderm cells by contacting the iPSCs with Activin A. In some embodiments, the iPSCs are further contacted with BMP4. In some embodiments, the iPSCs are contacted with a concentration of BMP4 that is, is about, is at least, is at least about, is not more than, or is not more than about, 1, 2, 3, 4, 5, 6, 7, 8, 9, 10, 11, 12, 13, 14, or 15 ng/mL of BMP4.

In some embodiments, the definitive endoderm cells are differentiated to foregut or hindgut spheroids. In some embodiments, the definitive endoderm cells are differentiated to foregut or hindgut spheroids by contacting the definitive endoderm cells with one or more (e.g. at least 1 or 2) of a GSK3 inhibitor or FGF4. In some embodiments, the GSK3 inhibitor is CHIR99021. In some embodiments, the FGF4 is recombinant FGF4. In some embodiments, the definitive endoderm cells are differentiated to foregut or hindgut spheroids without contacting the definitive endoderm cells with one or more (e.g. at least 1 or 2) of a GSK3 inhibitor or FGF4. In some embodiments, the definitive endoderm cells are differentiated to foregut or hindgut spheroids without contacting the definitive endoderm with CHIR99021 or FGF4, or both. In some embodiments, the definitive endoderm cells are differentiated to foregut or hindgut spheroids by contacting the definitive endoderm cells with epidermal growth factor (EGF). In some embodiments, the definitive endoderm cells are differentiated to foregut or hindgut spheroids by contacting the definitive endoderm cells with a BMP inhibitor. In some embodiments, the BMP inhibitor is Noggin. In some embodiments, the definitive endoderm cells are differentiated to foregut or hindgut spheroids by contacting the definitive endoderm cells with retinoic acid.

In some embodiments, the foregut or hindgut spheroids are embedded in a basement membrane or basement membrane mimetic or derivative thereof. In some embodiments, the foregut or hindgut spheroids are embedded in Matrigel. In some embodiments, the foregut or hindgut spheroids are cultured in basal gut medium (e.g. Minigut medium). In some embodiments, the foregut or hindgut spheroids are cultured in basal gut medium to differentiate the foregut or hindgut spheroids to gastrointestinal organoids. In some embodiments, basal gut medium comprises one or more of Advanced DMEM-F12, N2 supplement, B27 supplement, HEPES, L-glutamine, penicillin-streptomycin, epidermal growth factor (EGF), or a ROCK inhibitor (e.g. Y-27632), or any combination thereof. In some embodiments, basal gut medium comprises EGF.

In some embodiments, the definitive endoderm cells are differentiated to spheroids. In some embodiments, the definitive endoderm cells are differentiated to spheroids by contacting the definitive endoderm cells with one or more (e.g. at least 1, 2, 3, 4) of a GSK3 inhibitor, an FGF, BMP inhibitor, or retinoic acid (RA). In some embodiments, the GSK3 inhibitor is CHIR99021. In some embodiments, the FGF is FGF4. In some embodiments, the FGF4 is recombinant FGF4. In some embodiments, the BMP inhibitor is Noggin. In some embodiments, the definitive endoderm cells are differentiated to spheroids without contacting the definitive endoderm cells with one or more (e.g. at least 1, 2, 3, 4) of a GSK3 inhibitor, FGF4, BMP inhibitor, or RA, or any combination thereof. In some embodiments, the definitive endoderm cells are differentiated to spheroids without contacting the definitive endoderm with CHIR99021, FGF4, Noggin, or RA, or any combination thereof. In some embodiments, the definitive endoderm cells are differentiated to spheroids by contacting the definitive endoderm cells with epidermal growth factor (EGF).

### Preparation of Composite Organoids

Disclosed herein are composite organoids and methods of producing the same. In some embodiments, the methods comprise obtaining mono-dissociated mesenchymal cells isolated from one or more organoids, obtaining an epithelial structure isolated from an organoid or enteroid, combining the mono-dissociated mesenchymal cells and the epithelial structure, and culturing the combined mono-dissociated mesenchymal cells and the epithelial structure to form the composite organoid. In some embodiments, obtaining the mono-dissociated mesenchymal cells comprises isolating the mono-dissociated mesenchymal cells from the one or more organoids. In some embodiments, obtaining the epithelial structure comprises isolating the epithelial structure from the organoid or enteroid. The process of combining the mono-dissociated mesenchymal cells and epithelial structure from more than one organoid (or enteroid) enables the formation of organoids that comprise numbers of mesenchymal cells that are greater than what is typically achievable using conventional organoid differentiation methods known in the art. By performing these methods, desirable organoids, such as patient-derived organoids, or enteroids, which are devoid of mesenchyme, can be produced and cultured to more closely resemble biological tissue. In some embodiments, mono-dissociated mesenchymal cells are isolated from organoids of a same tissue type and can be combined with an epithelial structure isolated from an organoid of the same type to enrich the mesenchymal population of the organoid. In some embodiments, the increased number of mesenchymal cells improves the growth and maturation capability, both in vitro and when engrafted in vivo, thereby overcoming an issue that is commonly encountered with traditionally produced organoids.

In some embodiments, the methods provided herein allow for the formation of hybrid organoids comprising cell types of different organ tissue types (e.g. an organoid exhibiting both stomach and colonic cells). In some embodiments, mono-dissociated mesenchymal cells are isolated from organoids of different tissue types, while an epithelial structure can be isolated from an organoid of the same tissue type as one of the organoids used for mesenchymal cell isolation, or from a different tissue type from all of the other organoids. These hybrid organoids can be used in studies in personalized medicine, drug screening, or developmental biology, including high throughput studies. Also envisioned is the study of different aspects of cancer, such as migration, metastasis, vascularization, and immune evasion, using hybrid organoids comprising both normal and malignant cell types.

Also disclosed herein are composite organoids and compositions thereof, such as those produced by the methods disclosed herein. In some embodiments, the composite organoids comprise a mesenchyme and an epithelium. In some embodiments, the mesenchyme comprises mono-dissociated mesenchymal cells isolated from a first organoid. In some embodiments, the epithelium comprises an epithelial structure isolated from a second organoid or enteroid. In some embodiments, the composite organoid comprises a number of mesenchymal cells greater than the number of mesenchymal cells of the first organoid, or the second organoid or enteroid, or both. In some embodiments, the ratio of the number of mono-dissociated mesenchymal cells to epithelial cells in the composite organoid is greater than that of the first organoid, or second organoid or enteroid, or both. In some embodiments, the tissue type of the first organoid and the tissue type of the second organoid or enteroid is the same. In some embodiments, the composite organoid is a homogeneous organoid comprising one tissue type. In some embodiments, the tissue type of the first organoid and the tissue type of the second organoid or enteroid is different. In some embodiments, the tissue type of the first organoid from which the mesenchymal cells are isolated and the tissue type of the second organoid or enteroid from which the epithelial structure is isolated is different, where no repatterning of the epithelial cells by the mesenchymal cells occurs, such that the epithelium of the composite organoid maintains the tissue type of the second organoid or enteroid from which the epithelial structure is isolated. In some embodiments, no repatterning of the epithelial cells occurs when the epithelial structure is derived from an organoid that is at least 14, 15, 16, 17, 18, 19, 20, 21, 22, 23, 24, 25, 26, 27, 28, 29, or 30 days old or an organoid that is between 14-30, 15-30, 18-30, 15-20, or 15-25 days old. In some embodiments, no repatterning of the epithelial cells occurs when the epithelial structure is derived from an enteroid. In some embodiments, the enteroid is derived from adult tissue. In some embodiments, the tissue type of the first organoid from which the mesenchymal cells are isolated and the tissue type of the second organoid or enteroid from which the epithelial structure is isolated is different, where repatterning of the epithelial cells by the mesenchymal cells occurs, such that the epithelium of the composite organoid exhibits properties of the tissue type of the first organoid from which the mesenchymal cells are isolated. In some embodiments, repatterning of the epithelial cells by the mesenchymal cells may occur when the epithelial structure is derived from an organoid that is no more than 8, 9, 10, 11, 12, or 13 days old, or an organoid that is between 8-13, 8-10, or 10-13 days old. In some embodiments, enteroids are derived from adult tissue with well defined patterning, and epithelial structures derived from enteroids maintain their tissue type even when recombined with the mesenchymal cells. In some embodiments, the composite organoid is a heterogeneous organoid comprising more than one tissue type. In some embodiments, the composite organoid is the composite organoid produced by any one of the methods disclosed herein.

In some embodiments, the composite organoids disclosed herein may comprise one or more exogenous nucleic acids or proteins. For example, these one or more exogenous nucleic acids or proteins may be used as a reporter or marker.

In some embodiments, the composite organoids disclosed herein may comprise a genetic mutation. In some embodiments, the genetic mutation may be associated with a desired organ function or a reporter function. In some embodiments, the genetic mutation may be associated with a disease state or a model of a disease state. In some embodiments, a disease state or a model of a disease state may be induced in the composite organoids disclosed herein through other methods, such as treatment with a composition that induces the disease state or the model of the disease state.

The following describe the various aspects, which are typically, but not necessarily, included in the preparation of composite organoids.

### Source of Mesenchyme and Epithelial Cells

For any of the methods disclosed herein, 1) the one or more organoids from which the mono-dissociated mesenchymal cells are isolated, and 2) the organoid or enteroid from which the epithelial structure is isolated can be organoids of any tissue type, for example, brain, neuronal, muscle, thyroid, cardiac, pulmonary, kidney, bladder, testicular, pancreatic, gastrointestinal, esophageal, gastric, liver, intestinal, or colonic organoids or tissue types, or enteroids produced from intestinal or colonic (where it can also be called a colonoid) epithelial tissue. These donor organoids of any tissue type or enteroids can be produced according to any applicable methods known in the art. When produced by conventional methods, these organoids may exhibit little mesenchyme or reduced mesenchyme relative to biological tissue, which is counterproductive to long term tissue culture and/or engraftment into a subject, and may also be nonrepresentative of how the corresponding organ functions. In the case of enteroids, these lack any mesenchyme and are unamenable to culturing. Therefore, in some embodiments of the methods provided herein, any of these organoids or enteroids can be enriched for mesenchyme to improve these characteristics. It should be understood that the referred to “tissue type” of organoid is the type of tissue which the organoid most closely resembles based on considerations such as the phenotype(s) (e.g., gene expression, protein expression, morphology, etc.) of cells present in the organoid. The organoid need not be identical in all aspects to corresponding tissue to constitute an organoid of that “tissue type.”

Methods of producing these donor organoids or enteroids can be found, for example, in U.S. Pat. Nos. 9,719,068 and 10,174,289, and PCT Publications WO 2011/140411, WO 2015/183920, WO 2016/061464, WO 2017/192997, WO 2018/106628, WO 2018/200481, WO 2018/085615, WO 2018/085622, WO 2018/085623, WO 2018/226267, WO 2019/074793, WO 2020/023245, each of which is hereby expressly incorporated by reference in its entirety.

In some of the methods for producing the donor organoids or enteroids provided herein or known in the art, the donor organoids or enteroids are from defined sources. In some embodiments, the one or more organoids from which the mono-dissociated mesenchymal cells are isolated are derived from PSCs from a first subject. In some embodiments, the organoid or enteroid from which the epithelial structure is isolated is derived from PSCs or isolated from the first subject, or from a second subject that is not the first subject, e.g., from gastrointestinal tissue. In some embodiments, the PSCs are induced pluripotent stem cells. In some embodiments, the PSCs are obtained from the first subject and/or the second subject by reprogramming somatic or adult stem cells isolated from the first subject and/or the second subject. In some embodiments, the somatic or adult stem cells comprise bone marrow cells, peripheral cells, mobilized peripheral cells, or any other somatic cell. The somatic or adult stem cells can be reprogrammed to PSCs according to any applicable method conventionally known in the art. In some embodiments, the first subject and the second subject are mammals. In some embodiments, the first subject and the second subject are humans. In some embodiments, the first subject and the second subject are the same individual. In some embodiments, the first subject and/or the second subject have a disease, have previously had a disease, or are at risk of contracting a disease.

In some embodiments, the donor organoids or enteroids are first cultured for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 days, or any number of days within a range defined by any two of the aforementioned number of days, for example, 1 to 50 days, 10 to 30 days, 20 to 40 days, 1 to 30 days, or 20 to 50 days.

In some embodiments, the one or more organoids from which the mono-dissociated mesenchymal cells are isolated comprise a number of organoids that is, is about, is at least, is at least about, is not more than, or is not more than about, 1, 10, 10², 10³, 10⁴, 10⁵, 10⁶, 10⁷, 10⁸, or 10⁹ organoids, or any number of organoids within a range defined by any two of the aforementioned number of organoids, for example, 1 to 10⁹ organoids, 10² to 10⁷ organoids, 10⁴ to 10⁶ organoids, 1 to 10⁴ organoids, or 10⁴ to 10⁹ organoids.

In some embodiments, the organoids or enteroids are prepared in an extracellular matrix, or a mimetic or derivative thereof. Some examples of extracellular matrices, or mimetics or derivative thereof, include but are not limited to cell-based feeder layers, polymers, proteins, polypeptides, nucleic acids, sugars, lipids, poly-lysine, poly-ornithine, collagen, gelatin, fibronectin, vitronectin, laminin, elastin, tenascin, heparan sulfate, entactin, nidogen, osteopontin, basement membrane, Matrigel, hydrogel, PEI, WGA, or hyaluronic acid, or any combination thereof. In some embodiments, the extracellular matrix, or mimetic or derivative thereof, is or comprises Matrigel. In some embodiments, the organoids or enteroids are released from the extracellular matrix, or the mimetic or derivative thereof, when ready to be used. In some embodiments, the organoids or enteroids are released by depolymerizing the extracellular matrix, or the mimetic or derivative thereof. In some embodiments, the organoids or enteroids are released using Cell Recovery Solution (Corning). In some embodiments, where different tissue types of organoids or enteroids are used, the organoids or enteroids of different tissue types are processed simultaneously. In some embodiments, the organoids or enteroids have been previously cryopreserved. Cryopreserving the organoids or enteroids permits simultaneous processing, for example if the organoids or enteroids grow at different rates or are isolated and/or differentiated at different times.

In some embodiments, the organoids or enteroids are of more than one tissue type. In some embodiments, the organoids or enteroids of each tissue type may be cultured separately, for example, under conditions that are optimized for each tissue type. In other embodiments, the organoids or enteroids of each tissue type may be cultured together. In some embodiments, the organoids or enteroids of more than one tissue type comprise a number of tissue types that is, is about, is at least, is at least about, is not more than, or is not more than about, 2, 3, 4, 5, 6, 7, 8, 9, 10, 11, 12, 13, 14, 15, 16, 17, 18, 19, or 20 tissue types.

In some embodiments, the donor organoids or enteroids, or any of the precursor cells, are engineered to comprise one or more exogenous nucleic acids or proteins, or both. In some embodiments, the one or more exogenous nucleic acids or proteins may comprise a nucleic acid or protein having a desired organ function or a reporter function, such as a fluorescent or luminescent protein, or a nucleic acid encoding for a fluorescent or luminescent protein. In some embodiments, the donor organoids or enteroids are directly engineered to comprise the one or more exogenous nucleic acids or proteins. In some embodiments, the PSCs or gastrointestinal tissue that are used to derive the donor organoids or enteroids are engineered to comprise the one or more exogenous nucleic acids or proteins prior to forming the organoids or enteroids.

In some embodiments, the donor organoids or enteroids, or any of the precursor cells, either have or are engineered to comprise a genetic mutation. In some embodiments, the genetic mutation may be associated with a desired organ function or a reporter function. In some embodiments, the genetic mutation may be associated with a disease state or a model of a disease state. In some embodiments, the use of any one or more of these donor organoids or enteroids in the methods or composite organoid compositions disclosed herein may result in the composite organoids exhibiting all or some of the disease state or the model of the disease state, or symptoms thereof. The presence of the genetic mutation in the donor organoids or enteroids also generally apply to separate cellular components that make up the donor organoids or enteroids, including mesenchymal and epithelial cell populations. In some embodiments, the donor organoids or enteroids having the genetic mutation may be derived from a patient, such as one having or predisposed to a disease. In some embodiments, a disease state or a model of a disease state may be induced in the donor organoids or enteroids through other methods, such as treatment with a composition that induces the disease state or the model of the disease state.

Dissociation of Organoids into Mesenchymal Cells and Epithelial Structures

The donor organoids or enteroids produced according to any applicable method correspond to the one or more organoids from which the mono-dissociated mesenchymal cells are isolated and/or the organoid or enteroid from which the epithelial structure is isolated. Each of the donor organoids or enteroids are disrupted or dissociated to liberate the mono-dissociated mesenchymal cells or epithelial structures, which refer to fragments of organoid or enteroid epithelium that are not fully dissociated into single cells but are prepared as intact groups of epithelial cells. In some embodiments, the epithelial structure may contain additional cell types (e.g. mesenchymal cells).

For any of the methods disclosed herein, the one or more organoids from which the mono-dissociated mesenchymal cells are isolated, and the organoid or enteroid from which the epithelial structure is isolated each comprise a tissue type (brain, neuronal, muscle, thyroid, cardiac, pulmonary, kidney, bladder, testicular, pancreatic, gastrointestinal, esophageal, gastric, liver, intestinal, or colonic tissue types, or any combination thereof). In some embodiments, the tissue type of the one or more organoids from which the mono-dissociated mesenchymal cells are isolated and the tissue type of the organoid or enteroid are different. In some embodiments, the tissue type of the one or more organoids and the tissue type of the organoid or enteroid are the same.

The organoids or enteroids can be disrupted or dissociated using any applicable method conventionally known in the art, such as mechanical dissociation or enzymatic dissociation. In some embodiments, the mono-dissociated mesenchymal cells, or the epithelial structure, or both, are isolated by mechanical dissociation and filtration. In some embodiments, the organoids are mechanically dissociated using a pipette, microchannel, or other apparatus with an appropriately sized bore or channel to mechanically shear groups of cells without disrupting the individual cells. In some embodiments, the pipette is a conventional 5 mL pipette. In some embodiments, the appropriately sized bore or channel comprises a diameter. In some embodiments, the diameter is, is about, is at least, is at least about, is not more than, or is not more than about, 0.1, 0.5, 1, 1.5, 2, 2.5, 3, 3.5, 4, 4.5, or 5 mm in diameter, or any diameter within a range defined by any two of the aforementioned diameters. In some embodiments, the mesenchymal and epithelial components are further separated using a cell strainer. In some embodiments, the cell strainer has a mesh size that is, is about, is at least, is at least about, is not more than, or is not more than about, 40 μm, 70 μm, or 100 μm, or any mesh size within a range defined by any two of the aforementioned mesh sizes. In some embodiments, the cell strainer retains the epithelial structures while allowing the mono-dissociated mesenchymal cells to pass through. The separated epithelial structures and mono-dissociated mesenchymal cells can then be collected for further use.

In some embodiments, after dissociation, the collected mono-dissociated mesenchymal cells or the epithelial structures, or both, are cultured in a growth medium. In some embodiments, the growth medium is any medium disclosed herein or otherwise known in the art to support mesenchymal cells or epithelial cells, or both. In some embodiments, the growth medium comprises EGF, a ROCK inhibitor (e.g. Y-27632), or both. In some embodiments, the growth medium is Minigut media supplemented with hEGF or Y-27632, or both. In some embodiments, the culturing of the mono-dissociated mesenchymal cells or the epithelial structures, or both, permits the growth and/or expansion of the respective cells to obtained larger numbers of cells. In other embodiments, the mono-dissociated mesenchymal cells or the epithelial structures, or both, are used directly after dissociation.

In some embodiments, the isolated mono-dissociated mesenchymal cells or the epithelial structures, or both, are cryopreserved after dissociation. In some embodiments, the mono-dissociated mesenchymal cells or the epithelial structures, or both, are cryopreserved for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 days, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 days, or any number of days within a range defined by any two of the aforementioned times, for example, 1 to 120 days, 1 to 60 days, 10 to 120 days, 10 to 90 days, 10 to 60 days, 10 to 50 days, 20 to 30 days, 30 to 60 days, 60 to 120 days, 1 to 30 days, or 20 to 60 days. In some embodiments, the cryopreservation of the mono-dissociated mesenchymal cells or the epithelial structures, or both, allows for formation of composite organoids at a later point in time. In some embodiments, the composite organoid contains mesenchymal cells from multiple organoids and/or an epithelial structure that is from an organoid that is not the same organoid from which at least a portion of the mono-dissociated mesenchymal cells are isolated.

In some embodiments, the mono-dissociated mesenchymal cells or the epithelial structures isolated by any of the methods herein each are derived from organoids or enteroids of one or more tissue types. In some embodiments, the mono-dissociated mesenchymal cells or the epithelial structures derived from organoids or enteroids of more than one tissue type may be isolated from each of the organoids or enteroids of each tissue type after they are cultured separately. In some embodiments, the mono-dissociated mesenchymal cells isolated from separate organoids of each tissue type can then be pooled to provide a population of mono-dissociated mesenchymal cells of the more than one tissue type. In some embodiments, the epithelial structures isolated from separate organoids or enteroids of each tissue type can then be pooled to provide a population of epithelial structures of the more than one tissue type. In other embodiments, where the organoids or enteroids of each tissue type are cultured together, the dissociation of the organoids or enteroids results in populations of mono-dissociated mesenchymal cells and epithelial structures already comprised of more than one tissue type. In some embodiments, mono-dissociated mesenchymal cells or epithelial structures can be dissociated from separate populations of organoids that are cryopreserved and combined. In some embodiments, mono-dissociated mesenchymal cells or epithelial structures of a tissue type can be cryopreserved and later used to pool with one or more other, optionally cryopreserved, populations of mono-dissociated mesenchymal cells or epithelial structures, respectively. In other embodiments, mono-dissociated mesenchymal cells or epithelial structures of multiple tissue types can be pooled before cryopreservation. In some embodiments, each of the pooled mono-dissociated mesenchymal cells or the epithelial structures comprise a number of tissue types that is, is about, is at least, is at least about, is not more than, or is not more than about, 1, 2, 3, 4, 5, 6, 7, 8, 9, 10, 11, 12, 13, 14, 15, 16, 17, 18, 19, or 20 tissue types.

The dissociation steps provided herein are typically performed to isolate the mono-dissociated mesenchymal cells or epithelial structures, or both, from donor organoids or enteroids. However, intact donor organoids and/or enteroids can also be used for subsequent processes. In some embodiments, organoids comprising both a mesenchyme and epithelium and/or enteroids, which lack mesenchyme, can be used in lieu of the epithelial structure.

### Recombination of Mesenchyme and Epithelial Cells

After isolation, and optional pooling and/or cryopreservation, of the mono-dissociated mesenchymal cells and epithelial structures from donor organoids or enteroids, the mono-dissociated mesenchymal cells and an epithelial structure (or appropriate substitute, e.g. an intact organoid having mesenchyme and epithelium) are combined.

In one use, the mesenchyme (i.e. number of mesenchymal cells) can be enriched for any organoid. For organoid types that do not produce much mesenchyme (or less mesenchyme relative to in vivo tissue) when differentiated from conventional in vitro methods, it may be relatively difficult to culture and propagate such organoids. By performing the methods provided herein, organoids with robust amounts of mesenchyme can be produced. In some embodiments, the number of mesenchymal cells in the composite organoid is greater than the original number of mesenchymal cells of the one or more organoids from which the mono-dissociated mesenchymal cells are isolated, or the organoid or enteroid from which the epithelial structure is isolated, or both. In some embodiments, the number of mono-dissociated mesenchymal cells is, is about, is at least, is at least about, is not more than, or is not more than about, 1, 2, 3, 4, 5, 6, 7, 8, 9, 10, 20, 30, 40, 50, 60, 70, 80, 90, 100, 150, 200, 250, 300, 350, 400, 450, or 500 times, or any number within a range defined by any two of the aforementioned multiplicities, for example, 1 to 500 times, 10 to 400 times, 50 to 200 times, 1 to 100 times, or 100 to 500 times, the original number of mesenchymal cells of the one or more organoids or the organoid or enteroid, or both. In some embodiments, the ratio of the number of mono-dissociated mesenchymal cells to epithelial cells in the composite organoid is greater than that of the one or more organoids from which the mono-dissociated mesenchymal cells are isolated, or the organoid or enteroid from which the epithelial structure is isolated, or both. In some embodiments, the ratio of the number of mono-dissociated mesenchymal cells to epithelial cells in the composite organoid is, is about, is at least, is at least about, is not more than, or is not more than about, 1, 10, 10², 10³, 10⁴, 10⁵, 10⁶, 10⁷, 10⁸, or 10⁹ mesenchymal cells to epithelial cells, or any ratio of the number of mesenchymal cells to epithelial cells within a range defined by any two of the aforementioned numbers. In some embodiments, the total number of mono-dissociated mesenchymal cells per composite organoid is, is about, is at least, is at least about, is not more than, or is not more than about, 10⁴, 10⁵, 10⁶, or 10⁷ mono-dissociated mesenchymal cells, or any number of cells within a range defined by any two of the aforementioned number of mono-dissociated mesenchymal cells per composite organoid. Consequently, the resultant composite organoid has an enriched mesenchyme.

In some embodiments, the combination of the mono-dissociated mesenchymal cells and the epithelial structure can lead to either a homogeneous composite organoid or a heterogeneous composite organoid. In some embodiments, the mono-dissociated mesenchymal cells comprise only one tissue type and/or the one or more organoids from which the mono-dissociated mesenchymal cells are isolated comprise only one tissue type. In some embodiments, the epithelial structure comprises only one tissue type and/or the organoid or enteroid from which the epithelial structure is isolated comprises only one tissue type. In some embodiments, the one tissue type of the mono-dissociated mesenchymal cells and the epithelial structure is the same, such that the resultant composite organoid is a homogenous organoid comprising one tissue type. In some embodiments, the mono-dissociated mesenchymal cells comprise one or more tissue types and/or the one or more organoids from which the mono-dissociated mesenchymal cells are isolated comprises one or more tissue types. In some embodiments, the epithelial structure comprises one or more tissue types and/or the organoid or enteroid from which the epithelial structure is isolated comprises one or more tissue types. In some embodiments, the one or more tissue types of the mono-dissociated mesenchymal cells and the epithelial structures are different. In some embodiments, the tissue type of the mono-dissociated mesenchymal cells and the tissue type of the epithelial structure are different, where no repatterning of the epithelial structure by the mono-dissociated mesenchymal cells occurs, such that the epithelium of the composite organoid maintains the tissue type of the organoid or enteroid from which the epithelial structure is isolated. In some embodiments, no repatterning of the epithelial structure occurs when the epithelial structure is derived from an organoid that is at least 14, 15, 16, 17, 18, 19, 20, 21, 22, 23, 24, 25, 26, 27, 28, 29, or 30 days old, or an organoid that is between 14-30, 15-30, 18-30, 15-20, or 15-25 days old. In some embodiments, no repatterning of the epithelial structure occurs when the epithelial structure is derived from an enteroid. In some embodiments, the enteroid is derived from adult tissue. In some embodiments, the tissue type of the mono-dissociated mesenchymal cells and the tissue type of the epithelial structure are different, where repatterning of the epithelial structure by the mono-dissociated mesenchymal cells occurs, such that the epithelium of the composite organoid exhibits properties of the tissue type of the one or more organoids from which the mono-dissociated mesenchymal cells are isolated. In some embodiments, repatterning of the epithelial structure by the mono-dissociated mesenchymal cells may occur when the epithelial structure is derived from an organoid that is no more than 8, 9, 10, 11, 12, or 13 days old, or an organoid or enteroid that is between 8-13, 8-10, or 10-13 days old. In some embodiments, enteroids are derived from adult tissue with well defined patterning, and epithelial structures derived from enteroids maintain their tissue type even when recombined with the mono-dissociated mesenchymal cells. In some embodiments, the resultant composite organoid is a heterogeneous organoid comprising more than one tissue type. Homogeneous or heterogeneous composite organoids exhibiting properties of various organ types can be used in drug screening and studying biologically relevant functions and interactions of different organ tissues.

In some embodiments, an intact organoid comprising both a mesenchyme and an epithelium is used instead of an epithelial structure. The intact organoid comprising a tissue type and mono-dissociated mesenchymal cells comprising one or more tissue types are combined to produce a composite organoid comprising tissue types of both the intact organoid and the mono-dissociated mesenchymal cells. Accordingly, the composite organoid produced using an intact organoid may be either a homogeneous or heterogeneous organoid.

In some embodiments, an enteroid comprising only epithelial cells and no, or nearly no mesenchymal cells is used instead of an epithelial structure, or as a source for isolated epithelial structures. In some embodiments, the enteroid, or epithelial structure derived therefrom, is combined with mono-dissociated mesenchymal cells. In some embodiments, the tissue type of the enteroid and the mono-dissociated mesenchymal cells are both either intestinal or colonic tissue, or both. In other embodiments, the tissue type of the mono-dissociated mesenchymal cells comprises other tissue types other than intestinal or colonic tissue, thereby forming a composite organoid from an enteroid having properties of other tissue types. In some embodiments, the enteroid may be derived from intestinal or colonic tissue from a subject. In some embodiments, the enteroid is derived from intestinal or colonic tissue obtained from a biopsy. It is envisioned that formation of composite organoids from the processes herein using enteroids derived from intestinal or colonic tissue may be faster, easier, more cost-efficient, or less disruptive to the subject (e.g. if a biopsy has already been performed) compared to a similar organoid produced from subject-derived PSCs.

Any of the uses and/or embodiments provided herein may be used in combination with any other use and/or embodiment to produce the combined mono-dissociated mesenchymal cells and epithelial structures, and the resultant composite organoid.

### Combining the Mono-Dissociated Mesenchymal Cells and the Epithelial Structure

In some embodiments, the mono-dissociated mesenchymal cells and the epithelial structure (or appropriate substitute) are combined by centrifugation. In some embodiments, the parameters (e.g. speed and duration) for centrifugation suitable to combine the mono-dissociated mesenchymal cells and the epithelial structure is selected from a speed that is, is about, is not more than, is not more than about, is not less than, is not less than about 50, 100, 150, 200, 250, 300, 350, 400, 450, 500, 550, or 600×g, or a range defined by any two of the preceding values, for example 50-600×g, 100-300×g, 150-500×g, 200-400×g, 50-300×g, 50-350×g, or 250-600×g, and/or the duration of centrifugation is, is about, is not more than, is not more than about, is not less than, is not less than about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes, or a range defined by any two of the preceding values, for example 1-30 minutes, 1-5 minutes, 1-10 minutes, 10-20 minutes, 20-30 minutes, or 5-15 minutes. In other embodiments, the mono-dissociated mesenchymal cells and epithelial structure are combined by any other appropriate conventional method to aggregate cells, including allowing the cells to settle by gravity.

### Maturation of Composite Organoids

After the mono-dissociated mesenchymal cells and the epithelial structure (or any appropriate substitute or variant discussed herein) are combined, the combined mono-dissociated mesenchymal cells and the epithelial structure are cultured to form, grow, and mature the composite organoid. The combined cells may be cultured in vitro, or engrafted into a compatible organism (e.g. a human, mouse, rat, dog, cat, monkey, or any other mammal, which optionally may be immunocompromised) to mature.

In some embodiments, the combined mono-dissociated mesenchymal cells and the epithelial structure are cultured under growth conditions appropriate to support the resultant composite organoid. In some embodiments, the combined mono-dissociated mesenchymal cells and the epithelial structures are cultured under conditions described herein or otherwise known in the art. These conditions may comprise contacting the combined cells with one or more signaling pathway activators, signaling pathway inhibitors, or any other growth factors. For example, the combined cells are contacted with a ROCK inhibitor to improve cell survival. In some embodiments, the ROCK inhibitor is Y-27632. In some embodiments, the ROCK inhibitor is at a concentration that is, is about, is at least, is at least about, is not more than, or is not more than about, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 1 to 20 μM, 1 to 10 μM, 5 to 15 μM, or 10 to 20 μM. In some embodiments where the composite organoid comprises more than one tissue type, the conditions for culturing the combined cells may be optimized for one of the tissue types, or a combination of conditions optimized for more than one tissue types.

In some embodiments, the composite organoid is embedded in an extracellular matrix, or a mimetic or derivative thereof, for further culture and growth. In some embodiments, the composite organoid is cultured for a desired amount of time. In some embodiments, the composite organoid is cultured for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days, or any number of days within a range defined by any two of the aforementioned number of days, for example, 1 to 40 days, 1 to 10 days, 10 to 20 days, 20 to 30 days, 30 to 40 days, 1 to 2 days, 1 to 30 days, or 10 to 40 days.

In some embodiments, the composite organoid is engrafted to the appropriate region in a recipient subject. In some embodiments, the recipient subject is any one of the subjects from which the donor organoids or enteroids (and consequently the mono-dissociated mesenchymal cells and/or epithelial structures) are derived. In other embodiments, the recipient subject is not a subject from which the donor organoid or enteroids are derived. In some embodiments, the composite organoid may be engrafted after culturing the composite organoid for a certain amount of time, or directly after combining the mono-dissociated mesenchymal cells and epithelial structure.

In some embodiments, the engraftment is performed after culturing the composite organoid for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days, or any number of days of culture within a range defined by any two of the aforementioned days, for example, 1 to 50 days, 10 to 40 days, 20 to 30 days, 1 to 30 days, or 20 to 50 days. In some embodiments, the composite organoid is mature enough for engraftment and/or study a number of days before organoids prepared by other methods known in the art are at the same or similar mature state, wherein the number of days is, is about, is at least, is at least about, is not more than, or is not more than about, 1, 2, 3, 4, 5, 6, 7, 8, 9, 10, 11, 12, 13, 14, 15, 16, 17, 18, 19, or 20 days, or any number of days within a range defined by any two of the aforementioned number of days, for example, 1 to 20 days, 5 to 15 days, 10 to 15 days, 1 to 15 days, or 10 to 20 days.

In some embodiments, the composite organoid grows in the recipient subject for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 days, or any number of days within a range defined by any two of the aforementioned number of days, for example, 1-120 days, 1-60 days, 10-120 days, 10-90 days, 10-60 days, 10-30 days, 30-60 days, or 60-120 days. In some embodiments, the composite organoid exhibits integration with the recipient subject tissue. In some embodiments, the composite organoid comprises cell lineages of the tissue types of the constituent mono-dissociated mesenchymal cells and the epithelial structure. In some embodiments, the composite organoid develops cell lineages spontaneously after engraftment.

In some embodiments, the composite organoid exhibits greater engraftment and growth in the recipient subject compared to a comparable non-composite organoid or enteroid. In some embodiments, the comparable non-composite organoid or enteroid fails to engraft and/or grow in the recipient subject, but a composite organoid is able to successfully engraft and/or grow in the recipient subject. This may be use to the enrichment of mesenchyme cells in the composite organoid that are lacking in the comparable non-composite organoid. In some embodiments, the comparable non-composite organoid or enteroid is an organoid cultured in vitro according to applicable conventional methods known in the art or described herein (e.g. used for the donor organoids or enteroids) and engrafted into the same or similar recipient subject. In some embodiments, greater engraftment and growth can be measured in terms of time taken for full maturity after engraftment. In some embodiments, the composite organoids take a length of time that is, is about, is at least, is at least about, is not more than, or is not more than about, 5%, 10%, 20%, 30%, 40%, 50%, 60%, 70%, 80%, 90%, or 100% of the length of time taken for the applicable conventional organoid or enteroid to mature after engraftment, or any percentage of time within a range defined by any two of the aforementioned percentages of time, for example, 5% to 100%, 10% to 50%, 50% to 90%, or 30% to 50%. In some embodiments, greater engraftment and growth can be measured in terms of the relative size of the composite organoid. In some embodiments, the composite organoid is, is about, is at least, is at least about, is not more than, or is not more than about, 100%, 110%, 120%, 130%, 140%, 150%, 160%, 170%, 180%, 190%, 200%, 250%, 300%, 350%, 400%, 450% or 500% of the size (with respect to any dimension including but not limited to length, width, depth, radius, diameter, circumference, volume, or surface area) relative to the applicable conventional organoid, or any percentage of size within a range defined by any two of the aforementioned percentages, for example, 100% to 500%, 100 to 200%, 200% to 500%, or 300% to 500%.

### Gastrointestinal Organoids

One application of the composite organoids disclosed herein are for uses related to gastrointestinal organs. In some embodiments, the composite organoids disclosed herein are gastrointestinal organoids. In some embodiments, the gastrointestinal organoids are esophageal organoids, gastric organoids, fundic gastric organoids, antral gastric organoids, hepatic organoids, intestinal organoids, or colonic organoids, or any combination thereof. In some embodiments, the gastrointestinal organoids used in any of the methods disclosed herein (e.g. for isolating mono-dissociated mesenchymal cells and/or epithelial structures) are produced according to methods described herein or otherwise known in the art.

In some embodiments, 1) the one or more organoids from which the mono-dissociated mesenchymal cells are isolated and 2) the organoid or enteroid from which the epithelial structure is isolated each comprises a tissue type selected from an esophageal, gastric, hepatic, intestinal, or colonic tissue type, or any combination thereof. In some embodiments, the one or more organoids or the organoid or enteroid, or both, are human organoids or enteroids. In some embodiments, the one or more organoids or the organoid or enteroid, or both, comprise human esophageal organoids (HEOs), human gastric organoids (HGOs), human fundic gastric organoids (HFGOs), human antral gastric organoids (HAGOs), human hepatic organoids (HHOs), human intestinal organoids (HIOs), or human colonic organoids (HCOs), or any combination thereof.

In some embodiments, 1) the one or more organoids from which the mono-dissociated mesenchymal cells are isolated comprise an intestinal tissue type, and 2) the organoid or enteroid from which the epithelial structure is isolated comprises a tissue type selected from an esophageal, gastric, hepatic, intestinal, or colonic tissue type, or any combination thereof. In some embodiments, the one or more organoids from which the mono-dissociated mesenchymal cells are isolated are small intestinal organoids, optionally HIOs. The use of mono-dissociated mesenchymal cells isolated from intestinal organoids may offer some distinct advantages, as currently known processes for differentiating intestinal organoids from pluripotent stem cells produce substantial amounts of mesenchymal cells relative to differentiation protocols for organoids of other tissue types, which might result in reduced numbers of mesenchymal cells. In some embodiments, the methods of producing composite organoids disclosed herein are intended to compensate for this reduced differentiation of mesenchymal cells in other organoid protocols. In some embodiments, the large number of mesenchymal cells produced from intestinal organoid differentiation can be used to augment the organoids of other tissue types. However, it should be noted that the methods disclosed herein are not limited to using only intestinal organoids as the source of the mono-dissociated mesenchymal cells. Further, in some embodiments, the use of mono-dissociated mesenchymal cells as disclosed herein has additional/other advantages, such as producing composite organoids of one tissue type.

In some embodiments, the tissue type of the one or more organoids and the tissue type of the organoid or enteroid are different. In some embodiments, the resulting composite gastrointestinal organoid is a heterogeneous organoid. As applied to the composite organoids disclosed herein, the composite organoids may comprise a mesenchyme comprising a first tissue type selected from an esophageal, gastric, hepatic, intestinal or colonic tissue type, or any combination thereof, and an epithelium comprising a second tissue type selected from an esophageal, gastric, hepatic, intestinal, or colonic tissue type, or any combination thereof. In some embodiments, the first tissue type and second tissue type have at least one difference in tissue types. In some embodiments, some non-limiting examples of composite gastrointestinal organoids are intestinal-mesenchyme/intestinal-epithelium organoids, intestinal-mesenchyme/colonic-epithelium organoids, intestinal-mesenchyme/gastric-epithelium organoids, intestinal-mesenchyme/enteroid-epithelium organoids, gastric-mesenchyme/gastric-epithelium organoids, and colonic-mesenchyme/colonic-epithelium organoids. In some embodiments, the tissue type of the one or more organoids from which the mono-dissociated mesenchymal cells are isolated and the tissue type of the organoid or enteroid from which the epithelial structure is isolated are different, where no repatterning of the epithelial structure by the mono-dissociated mesenchymal cells occurs, such that the epithelium of the composite organoid maintains the tissue type of the organoid or enteroid from which the epithelial structure is isolated. In some embodiments, no repatterning of the epithelial structure occurs when the epithelial structure is derived from an organoid that is at least 14, 15, 16, 17, 18, 19, 20, 21, 22, 23, 24, 25, 26, 27, 28, 29, or 30 days old, or an organoid that is between 14-30, 15-30, 18-30, 15-20, or 15-25 days old. In some embodiments, no repatterning of the epithelial structure occurs when the epithelial structure is derived from an enteroid. In some embodiments, the enteroid is derived from adult tissue. In some embodiments, the tissue type of the one or more organoids from which the mono-dissociated mesenchymal cells are isolated and the tissue type of the organoid or enteroid from which the epithelial structure is isolated are different, where repatterning of the epithelial structure by the mono-dissociated mesenchymal cells occurs, such that the epithelium of the composite organoid exhibits properties of the tissue type of the one or more organoids from which the mono-dissociated mesenchymal cells are isolated. In some embodiments, repatterning of the epithelial structure by the mono-dissociated mesenchymal cells may occur when the epithelial structure is derived from an organoid that is no more than 8, 9, 10, 11, 12, or 13 days old, or an organoid that is between 8-13, 8-10, or 10-13 days old. In some embodiments, enteroids are derived from adult tissue with well-defined patterning, and epithelial structures derived from enteroids maintain their tissue type even when recombined with the mono-dissociated mesenchymal cells.

In some embodiments, the tissue type of the one or more organoids and the tissue type of the organoid or enteroid are the same. In some embodiments, the one or more organoids and the organoid or enteroid each comprises only one of esophageal, gastric, hepatic, intestinal, or colonic tissue types. In some embodiments, the resultant composite gastrointestinal organoid is a homogeneous organoid comprising only one tissue type.

In some embodiments, the combined mono-dissociated mesenchymal cells and the epithelial structures are cultured under conditions optimized for esophageal, gastric, hepatic, intestinal, or colonic organoid growth. In some embodiments, the combined mono-dissociated mesenchymal cells and the epithelial structures are cultured in Minigut media. In some embodiments, the Minigut media comprises one or more of Advanced DMEM/F12 medium, glutamine, HEPES, penicillin, streptomycin, N2 supplement, B27 supplement, epithelial growth factor (EGF), or a ROCK inhibitor, or any combination thereof. In some embodiments, the EGF is human EGF (hEGF). In some embodiments, the EGF is at a concentration that is, is about, is at least, is at least about, is not more than, or is not more than about, 10, 20, 30, 40, 50, 60, 70, 80, 90, 100, 110, 120, 130, 140, 150, 160, 170, 180, 190, or 200 ng/mL, or any concentration within a range defined by any two of the aforementioned concentrations, for example, 10 to 200 ng/mL, 50 to 150 ng/mL, 80 to 120 ng/mL, 10 to 100 ng/mL, or 100 to 200 ng/mL. In some embodiments, the ROCK inhibitor is Y-27632. In some embodiments, the ROCK inhibitor is at a concentration that is, is about, is at least, is at least about, is not more than, or is not more than about, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 1 to 20 μM, 1 to 10 μM, 5 to 15 μM, or 10 to 20 μM.

In some embodiments, the composite gastrointestinal organoid comprises a lumen. In some embodiments, the composite gastrointestinal organoid comprises a lumen that occupies a percentage of the total volume of the composite gastrointestinal organoid. In some embodiments, the lumen occupies a percentage of the total volume of the composite gastrointestinal organoid that is, is about, is at least, is at least about, is not more than, or is not more than about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 38%, 39%, or 40% of the total volume of the composite gastrointestinal organoid, or any percentage within a range defined by any two of the aforementioned percentages, for example, 1% to 40%, 10% to 30%, 15% to 20%, 1% to 20%, or 10% to 40%.

In a non-limiting example of an HIO-mesenchyme/HCO-epithelium heterogeneous organoid, the heterogeneous organoid expresses sucrase-isomaltase (SI), which is specific for the epithelium of the small intestine, and does not express SATB2, which is specific for the epithelium of the large intestine.

In a non-limiting example of an HIO-mesenchyme/HEO-epithelium heterogeneous organoid, the heterogeneous organoid expresses KRT5, KRT14, KRT13, IVL, p63, and CDH1.

### Uses of Composite Organoids

The composite organoids disclosed herein or produced by any of the methods disclosed herein may be used for various purposes including but not limited to providing a source of tissue for transplant, drug screening, study of organ function, neurological function, microbiome interaction, or any combination thereof.

In some embodiments, the methods disclosed herein comprise the additional step of transplanting any one of the composite organoids disclosed herein into a recipient subject, not only to mature the organoid as described herein, but in addition or alternatively to restore, repair or improve an organ function in the recipient subject. These methods may be used to treat a subject having compromised organ function, or ameliorated, inhibiting, or treating a detrimental organ disorder in a subject in need thereof. In some embodiments, the recipient subject is the subject from which the one or more organoids from which the mono-dissociated mesenchymal cells are isolated, or the organoid or enteroid from which the epithelial structure is isolated, or both. In some embodiments, the composite organoid or a component thereof is derived from PSCs isolated from the recipient subject. In some embodiments, the recipient subject is a mammal. In some embodiments, the recipient subject is an immunodeficient mammal. In some embodiments, the recipient subject is an immunodeficient mouse. In some embodiments, the recipient subject is a monkey, cat, dog, hamster, or rat. In some embodiments, the recipient subject is an immunocompromised monkey, cat, dog, hamster, or rat. In some embodiments, the recipient subject is a human. In some embodiments, the recipient subject is an immunocompromised human. In some embodiments, the recipient subject is an immunocompetent human. In some embodiments, the recipient subject is an immunocompetent human treated with immunosuppressants. In some embodiments, the recipient subject is an immunocompetent human and the composite organoid is autologous to the recipient subject. In some embodiments, the recipient subject is an immunocompetent human and the composite organoid is allogeneic to the recipient subject. In some embodiments, the recipient subject is a mammal that is in need of an organ transplant. In some embodiments, the recipient subject is a human that is in need of an organ transplant.

Also described herein are composite organoids for use in treating a malady in a subject in need thereof. In some embodiments, the composite organoids are the composite organoids described herein. In some embodiments, the composite organoids are the composite organoids produced by any one of the methods described herein.

In any one of the methods of treatment or uses provided herein, the composite organoid can be a composite gastrointestinal organoid. Accordingly, any one of the methods provided herein applies to treating a subject having compromised gastrointestinal function, or ameliorating, inhibiting, or treating a detrimental gastrointestinal disorder in a subject in need thereof. In some embodiments, the methods comprise transplanting or engrafting a composite gastrointestinal organoid into the subject. In some embodiments, the composite gastrointestinal organoid is an esophageal organoid, gastric organoid, fundic gastric organoid, antral gastric organoid, hepatic organoid, small intestinal (intestinal) organoid, or large intestinal (colonic) organoid. In some embodiments, the subject is in need of a gastrointestinal transplant. In some embodiments, the gastrointestinal organoid is transplanted or engrafted as a whole gastrointestinal organoid. In some embodiments, the transplant site is a gastrointestinal tissue. The composite gastrointestinal organoids may also be used for treating a gastrointestinal malady in a subject in need thereof.

Also disclosed herein are methods for screening for a candidate therapeutic. In some embodiments, the methods comprise contacting any one of the organoids disclosed herein with the candidate therapeutic and determining the effect of the candidate therapeutic on the organoid. In some embodiments, the organoid is genetically modified. In some embodiments, the organoid is genetically modified to exhibit a disease or model thereof. In some embodiments, the mesenchyme and/or the epithelium of the organoid is genetically modified. In some embodiments, the mesenchyme and/or the epithelium of the organoid is genetically modified to exhibit a disease or model thereof.

### EXAMPLES

Some aspects of the embodiments discussed herein are disclosed in further detail in the following examples, which are not in any way intended to limit the scope of the present disclosure. Those in the art will appreciate that many other embodiments also fall within the scope of the disclosure, as it is described herein and in the claims.

### Example 1. Producing Composite Organoid Compositions

Human pluripotent stem cells were cultured and induced to differentiate into definitive endoderm cells. Subsequently, the definitive endoderm cells were differentiated into human intestinal organoids (HIO), human colonic organoids (HCO), or human antral gastric organoids (HAGO). Organoids after day 10 to 30 of culture can be used for the following steps. Alternatively, enteroids, which lack mesenchyme, can be derived from intestinal or colonic tissue from a subject. Methods of producing these organoids or enteroids can be found, for example, in PCT Publications WO 2011/140441, WO 2015/183920, WO 2016/061464, WO 2017/192997, and WO 2018/106628, each of which is hereby expressly incorporated by reference in its entirety.

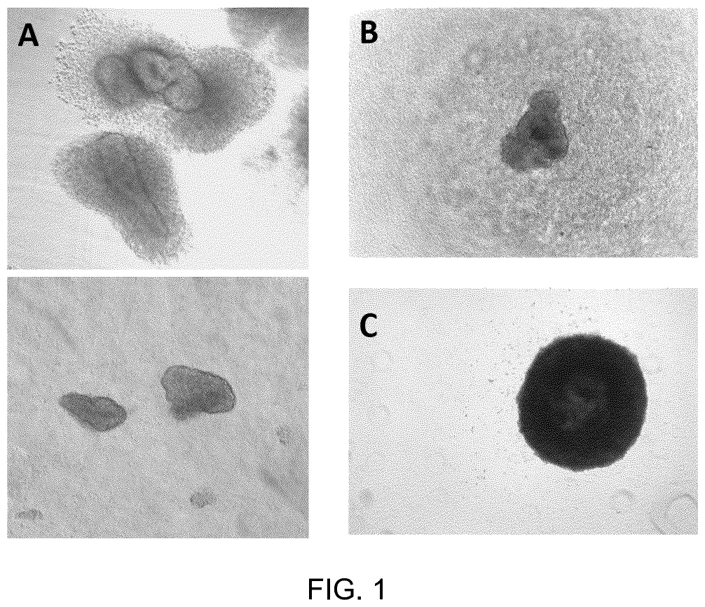

Organoids or enteroids were prepared in an extracellular matrix (e.g. Matrigel Growth Factor Reduced [Corning]). The organoids or enteroids were washed with an appropriate volume of Dulbecco's PBS (DPBS; e.g. 500 μL for 24-well plate wells), the DPBS was removed, and an appropriate volume of ice-cold Cell Recovery Solution (Corning; e.g. 500 μL) was added to detach the extracellular matrix drops from the culture plate. The organoids or enteroids and extracellular matrix were transferred to a 15 mL tube and incubated at 4° C. for 30 minutes with gentle agitation to induce depolymerization of the extracellular matrix. Up to 24 wells can be pooled in the same 15 mL tube. For hetero-recombination purposes, the different types of organoids or enteroids are typically processed at the same time in separate tubes. The tube was centrifuged at 300×g for 5 minutes, the supernatant was discarded, and 5 mL of fresh ice-cold Cell Recovery Solution was added. The mesenchyme and the epithelium of the organoids (or epithelium of the enteroids) were mechanically dissociated by pipetting up and down using a 5 mL serological pipette. The dissociation process was monitored regularly under a microscope. Complete dissociation was achieved when intact epithelial structures are free from surrounding mesenchyme (FIG. 1, panel A). To facilitate the separation of the mesenchyme from the epithelium and prevent the epithelial structures from breaking apart, manual dissociation can be alternated with incubations at 4° C. for 10 minutes with gentle agitation. The dissociated solution was filtered using a 40 μm mesh size cell strainer placed upside-down on top of a 50 mL tube to separate the mono-dissociated mesenchyme from the epithelial structures. The cell strainer was rinsed with 5 mL of cold fresh DPBS. Using forceps, the cell strainer retaining epithelial structures was transferred right side up to a 6 well plate containing 5 mL of DPBS. The cell strainer was dipped into the solution several times to detach the epithelial structures from the cell strainer mesh into the well. To prevent the epithelial structures from remaining on the cell strainer mesh and from attaching to the bottom of the well, the cell strainer can be soaked in a solution of 0.5% BSA in PBS in the well for 30 minutes before use. The 50 mL tube containing the mono-dissociated mesenchyme was centrifuged at 300×g for 5 minutes, the supernatant was discarded, and 2 mL of Minigut media (Advanced DMEM/F12 medium supplemented with 2 mM glutamine, 10 mM HEPES, 100 U/mL penicillin, 100 μg/mL streptomycin, lx N2 supplement, lx B27 supplement) supplemented with 100 ng/mL human epithelial growth factor (hEGF) and 10 μM Y-27632 (or equivalent ROCK inhibitor). The number of mesenchymal cells are counted using trypan blue to exclude any dead cells. If epithelial cell contamination is observed in the mesenchymal cell solution, the solution in the tube can be transferred to a plate and incubated for several minutes at room temperature to allow the epithelial cells to adhere to the bottom of the plate. The non-adhered mesenchymal cells can then be slowly aspirated and used for subsequent steps.

150,000 mesenchymal cells per well are seeded to an Ultra-Low Attachment round bottom 96 well plate (Corning). Under a horizontal laminar flow hood, the epithelial structures are picked up with a micropipette. One epithelial structure is added to each well containing mesenchymal cells. Epithelial structures may be from the same type of organoid as the mesenchymal cells, a different type of organoid as the mesenchymal cells, or an enteroid depending on the purpose of the experiment. If the epithelial structures are larger than the end of the micropipette tip, the bore of the tip can be expanded by cutting off the end using sterile scissors. The plate is centrifuged at 300×g for 2 minutes to aggregate the epithelial structure and mesenchymal cells (FIG. 1, panel B). The plate is incubated overnight at 37° C., upon which the combined cells form an organoid morphology (FIG. 1, panel C). Each composite organoid is collected using a micropipette with an appropriate size bore and plated in 50 μL Matrigel drops in a 24 well plate. To the plate is added 500 μL of Minigut media supplemented with 100 ng/mL human EGF. The composite organoids are maintained in culture for the desired amount of time. In some embodiments, the composite organoids are cultured for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days, or any number of days within a range defined by any two of the aforementioned number of days, for example, 1 to 40 days, 1 to 10 days, 10 to 20 days, 20 to 30 days, 30 to 40 days, 1 to 2 days, 1 to 30 days, or 10 to 40 days.

### Example 2. Recombination of Epithelium and Mesenchyme from Different Sources Robustly Produce Functional Heterogeneous Organoids

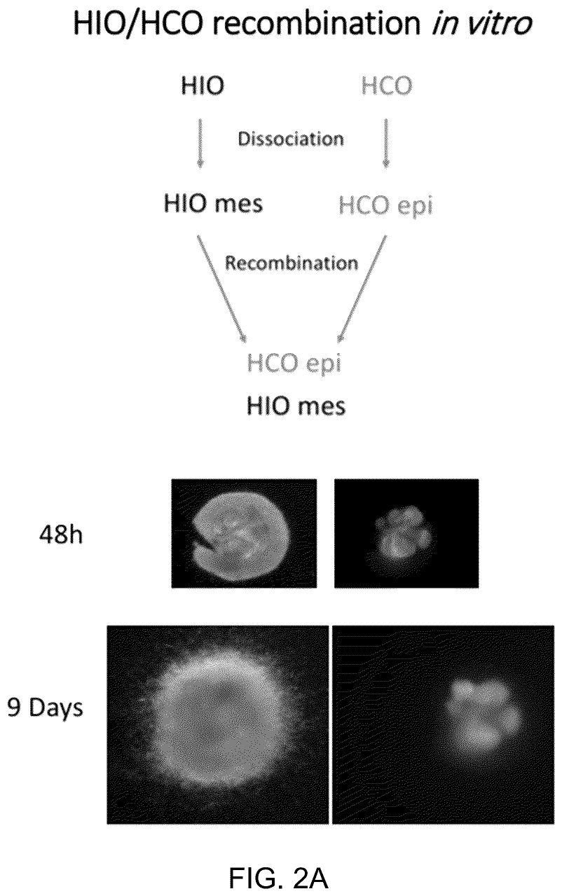

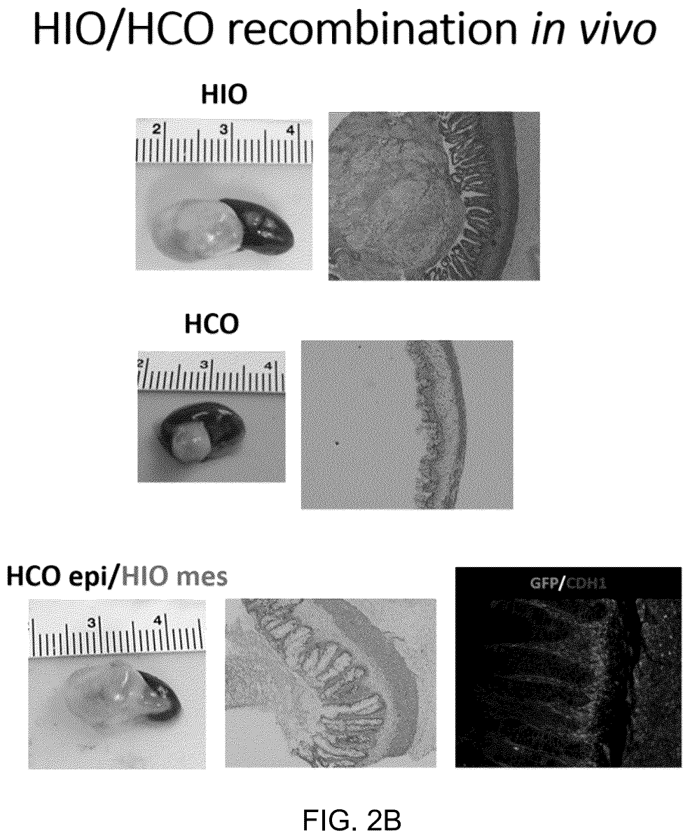

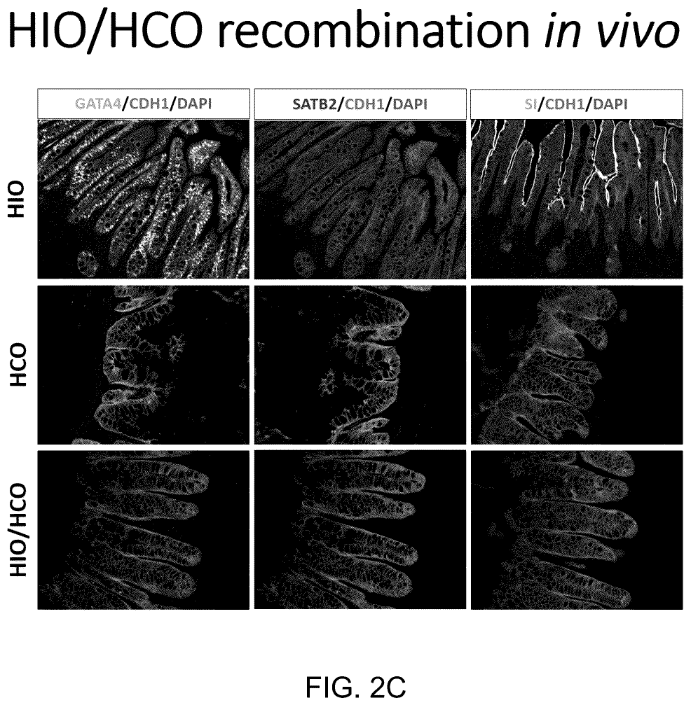

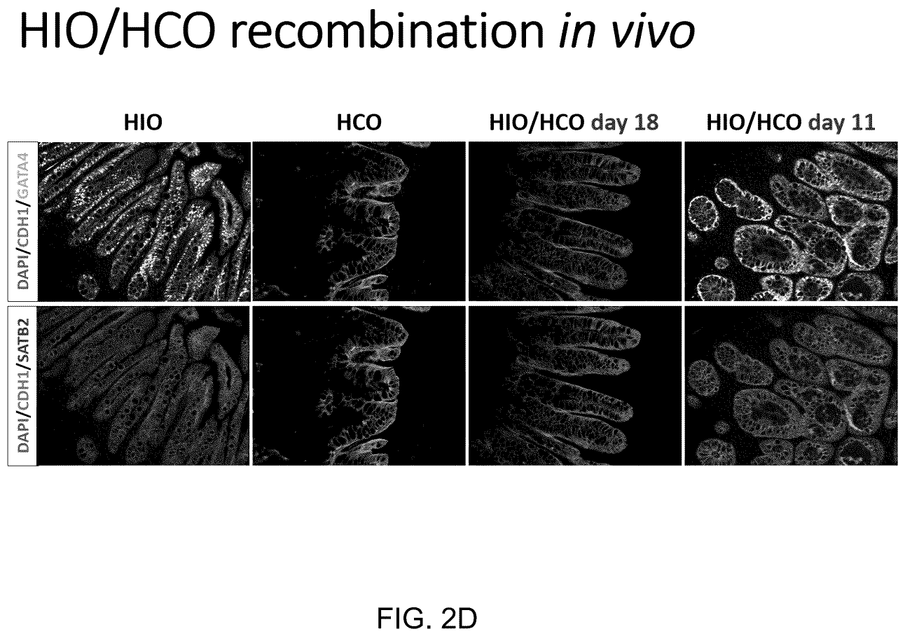

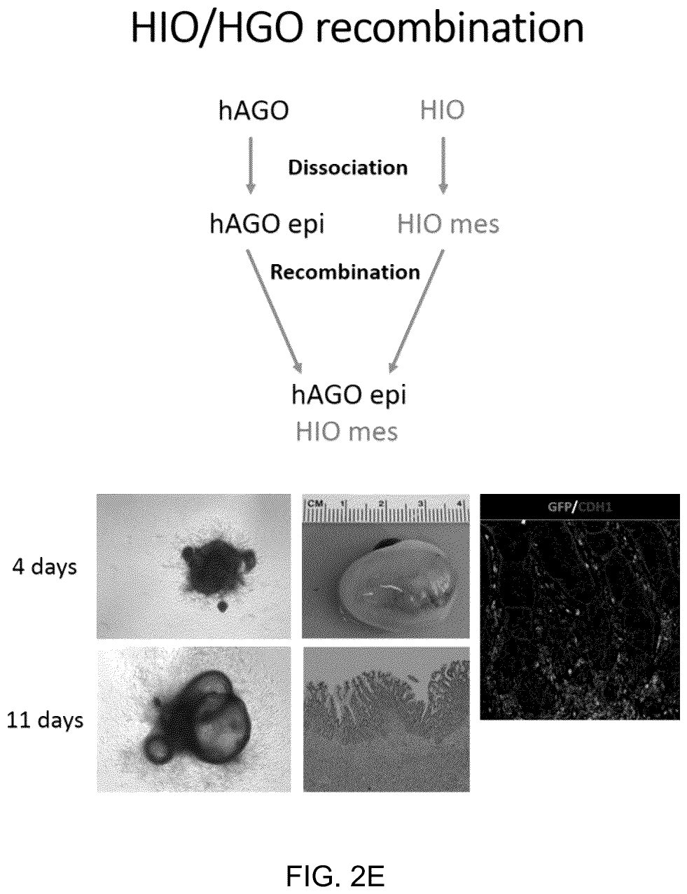

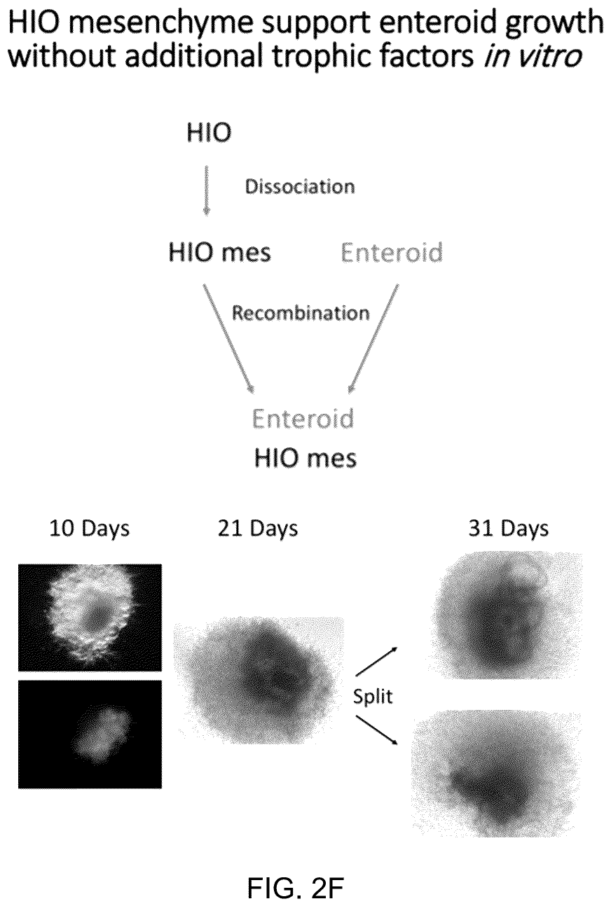

Human intestinal organoids (HIOs) and human colonic organoids (HCOs) were prepared separately. The HCOs were differentiated from GFP-expressing iPSCs. Mono-dissociated mesenchyme of the HIOs and epithelial structures of the HCOs were prepared and recombined according to Example 1 (using either day 11 or day 18 organoids) to form HIO-mesenchyme/HCO-epithelium heterogeneous organoids. These organoid exhibited robust recombination and growth in vitro when checked at 48 hours and 9 days following recombination, with intestinal mesenchyme encapsulating GFP-positive colonic epithelium (FIG. 2A). After engraftment onto the kidney capsule of immunodeficient mice, the heterogeneous organoid prepared from day 18 source organoids matured and form a luminal structure and distinct microvilli (FIG. 2B). The microvilli, originating from the progenitor colonic epithelium, were positive for GFP and E-cadherin (E-CAD; CDH1). The heterogeneous organoid prepared from day 18 source organoids also expressed special AT-rich sequence binding protein 2 (SATB2) in the epithelial layer but were negative for small intestine-specific sucrase-isomaltase (SI), suggesting that the epithelial structures from these HCOs maintained their distal characteristic (FIG. 2C).

The heterogeneous organoid prepared from day 11 source organoids expressed small intestine-specific GATA binding protein 4 (GATA4) but were negative for colon-specific SATB2, demonstrating the interesting property that younger and more immature epithelial structures can be reprogrammed to exhibit properties mediated by the surrounding mesenchyme (in this case, the HCO-derived epithelium exhibits small intestine properties) (FIG. 2D).

HIOs and human gastric organoids (HGOs) were prepared separately. Mono-dissociated mesenchyme of the HIOs and epithelial structures of the HGOs were prepared and recombined according to Example 1 to form HIO-mesenchyme/HGO-epithelium heterogeneous organoids. These organoid exhibited robust recombination and growth in vitro when checked at 4 days and 11 days following recombination (FIG. 2E).

HIOs and human enteroids were prepared separately. As the enteroids lack mesenchyme, further processing was not necessary. Mono-dissociated mesenchyme of the HIOs and the enteroids were recombined according to Example 1 to form a HIO-mesenchyme/enteroid heterogeneous organoids. These organoids exhibited robust recombination and growth in vitro when checked 10 days, 21 days, and 31 days following recombination (FIG. 2F).

### Example 3. Engraftment of Composite Organoids

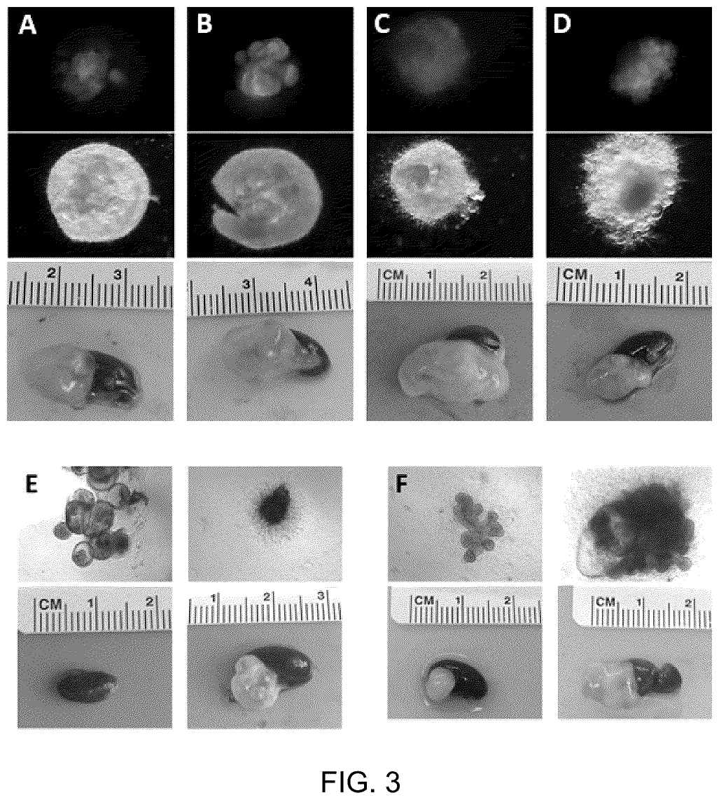

Various heterogeneous organoids were assessed for engraftment potential into immunocompromised mouse models. Human organoids differentiated from iPSCs to different organ types have been observed to have variable amounts of mesenchyme. For example, while HIOs produced according to known methods have ample mesenchymal cells to support epithelium and organoid maturation, HGOs and HCOs have significantly reduced numbers of mesenchymal cells and enteroids completely lack mesenchyme, resulting in difficulties in engraftment of these organoid types. The process of recombining mesenchymal and epithelial components from separate organoid sources at mesenchymal to epithelial ratios that more closely resemble in vivo tissue results in greater success of organoid engraftment and growth when transplanted to mouse kidney capsules (FIG. 3). HIO-mesenchyme/HIO-epithelium, HIO-mesenchyme/HCO-epithelium, and HIO-mesenchyme/HAGO-epithelium heterogeneous organoids all exhibited successful engraftment and maturation. HIO-mesenchyme/intestinal enteroid organoids also were engraftable. Furthermore, HAGO-mesenchyme/HAGO-epithelium and HCO-mesenchyme/HCO-epithelium homogenous organoids were prepared by isolating mesenchymal cells from several iPSC-differentiated HAGO or HCO, respectively, and recombining these mesenchymal cells with HAGO or HCO epithelial structures, thereby enriching the number of supporting mesenchymal cells available in each organoid. Compared to control HAGO and HCO organoids, which showed no or limited maturation and growth following engraftment, the mesenchyme-enriched HAGO and HCO organoids grew more robustly on the recipient kidney capsule tissue.

### Example 4. Production of Composite Esophageal Organoids

Like HGO and HCOs, human esophageal organoids (HEOs) produced by previous methods have reduced numbers of mesenchymal cells compared to intestinal organoids. Methods of producing esophageal organoids can be found, for example, in PCT Publication WO 2019/074793, which is hereby expressly incorporated by reference in its entirety.

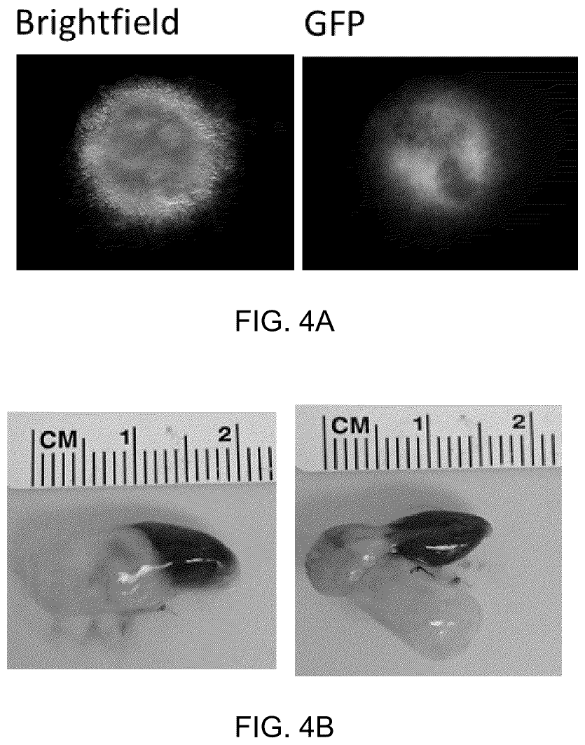

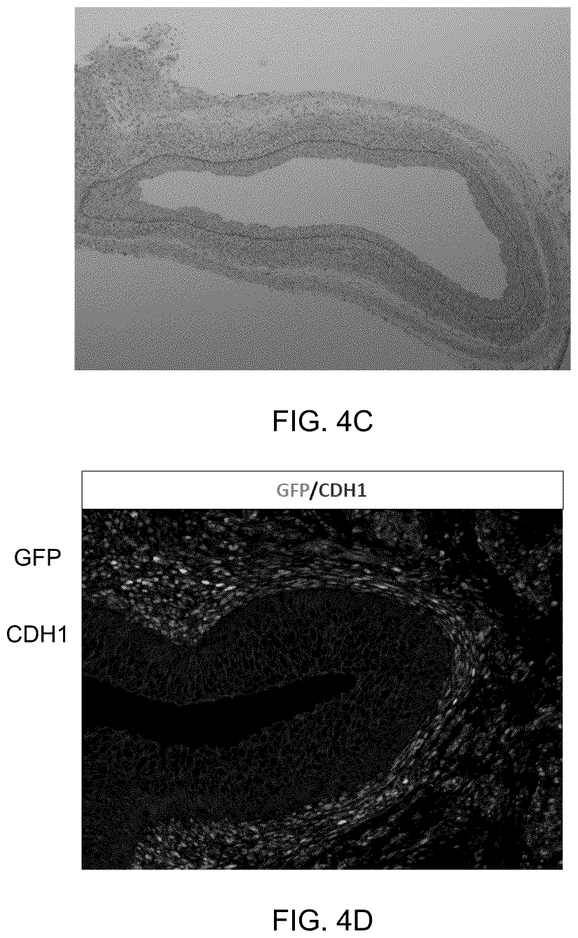

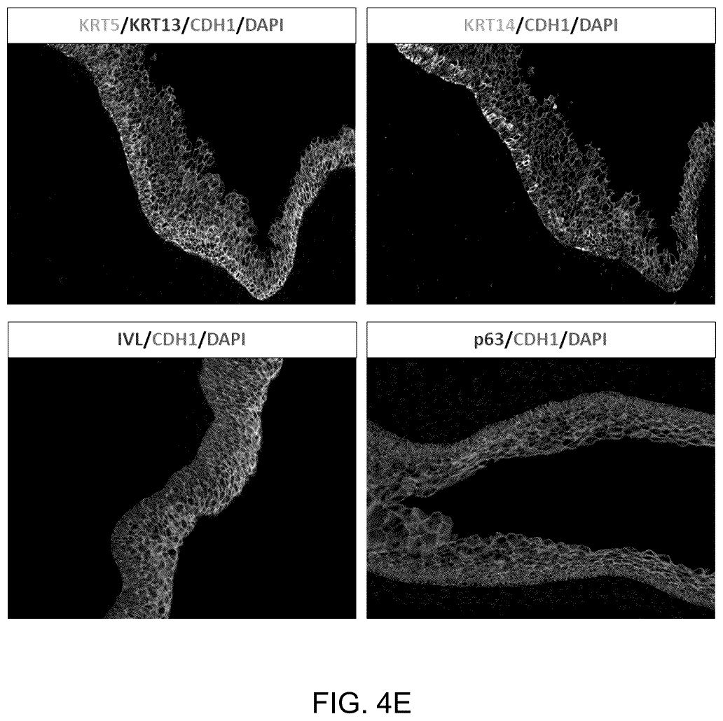

Mono-dissociated HIO mesenchyme (˜50,000 cells) and intact HEO epithelium (1 or 2 structures) were recombined at day 12 in low attachment 96 well plates used the protocols described herein. The next day, recombined HIO/HEO were transferred into Matrigel and cultured in vitro until transplantation under the kidney capsule of NSG mice at day 28. Transplanted tissues were harvested 8 weeks after engraftment, fixed in 4% paraformaldehyde, processed, and embedded in paraffin for imaging. FIG. 4A depicts images of combined organoid structures made up of the HIO mesenchyme (which are GFP positive) and HEO epithelium (which are GFP negative) 48 hours after the recombination procedure. FIG. 4B depicts images of HIO-mesenchyme/HEO-epithelium organoids after 8 weeks following transplantation into mice kidney capsules. FIG. 4C depicts hematoxylin/eosin staining of a transplanted HIO-mesenchyme/HEO-epithelium organoid. FIG. 4D depicts images of GFP (denoting HIO mesenchyme) and E-cadherin (CDH1; denoting HEO epithelium) showing a distinct isolation of the two layers. FIG. 4E depicts immunofluorescence images showing expression of keratin 5 (KRT5), keratin 14 (KRT14), keratin 13 (KRT13), involucrin (IVL), p63, and CDH1 in the mature esophageal epithelium.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” is typically interpreted as “including but not limited to,” the term “having” is typically interpreted as “having at least,” the term “includes” is typically interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases is typically construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” is typically interpreted to mean “at least one” or “one or more”); the same holds true for the use of definite articles used to introduce claim recitations. In addition, even if a specific number of an introduced claim recitation is explicitly recited, those skilled in the art will recognize that such recitation is typically interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, is typically understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

### REFERENCES

K. W. McCracken, Mechanisms of endoderm patterning and directed differentiation of human stem cells into foregut tissues, PhD Thesis, University of Cincinnati, 2014.
J. O. Mnnera, N. Sundaram, S. A. Rankin, D. Hill, C. Watson, M. Mahe, J. E. Vallance, N. F. Shroyer, K. L. Sinagoga, A. Zarzoso-Lacoste, J. R. Hudson, J. C. Howell, P. Chatuvedi, J. R. Spence, J. M. Shannon, A. M. Zorn, M. A. Helmrath, J. M. Wells, Differentiation of Human Pluripotent Stem Cells into Colonic Organoids via Transient Activation of BMP Signaling, Cell Stem Cell. 21 (2017) 51-64.e6. https://doi.org/10.1016/j.stem.2017.05.020.
C. L. Watson, M. M. Mahe, J. Mnnera, J. C. Howell, N. Sundaram, H. M. Poling, J. I. Schweitzer, J. E. Vallance, C. N. Mayhew, Y. Sun, G. Grabowski, S. R. Finkbeiner, J. R. Spence, N. F. Shroyer, J. M. Wells, M. A. Helmrath, An in vivo model of human small intestine using pluripotent stem cells, Nat. Med. 20 (2014) 1310-1314. https://doi.org/10.1038/nm.3737.
M. M. Mahe, N. Sundaram, C. L. Watson, N. F. Shroyer, M. A. Helmrath, Establishment of human epithelial enteroids and colonoids from whole tissue and biopsy, JoVE J. Vis. Exp. (2015) e52483.
M. M. Mahe, N. E. Brown, H. M. Poling, M. A. Helmrath, In vivo model of small intestine, in: Organ Regen., Springer, 2017: pp. 229-245.

What is claimed is:

1. A method of producing a composite organoid, comprising:
a) obtaining mono-dissociated mesenchymal cells isolated from one or more organoids;
b) obtaining an epithelial structure isolated from an organoid or enteroid;
c) combining the mono-dissociated mesenchymal cells and the epithelial structure; and
d) culturing the combined mono-dissociated mesenchymal cells and the epithelial structure to form the composite organoid.

2. The method of claim 1, wherein obtaining the mono-dissociated mesenchymal cells comprises isolating the mono-dissociated mesenchymal cells from the one or more organoids.

3. The method of claim 1 or 2, wherein obtaining the epithelial structure comprises isolating the epithelial structure from the organoid or enteroid.

4. The method of any one of claims 1-3, wherein the mono-dissociated mesenchymal cells, or the epithelial structure, or both, are isolated by mechanical dissociation and filtration.

5. The method of any one of the preceding claims, wherein the mono-dissociated mesenchymal cells and the epithelial structure are combined by centrifugation.

6. The method of any one of the preceding claims, wherein the number of mesenchymal cells in the composite organoid is greater than the original number of mesenchymal cells of the organoid or enteroid from which the epithelial structure is isolated, such that the composite organoid has an enriched mesenchyme.

7. The method of any one of the preceding claims, wherein 1) the one or more organoids from which the mono-dissociated mesenchymal cells are isolated and 2) the organoid or enteroid from which the epithelial structure is isolated each comprises a tissue type selected from an esophageal, gastric, hepatic, intestinal, or colonic tissue type, or any combination thereof.

8. The method of any one of the preceding claims, wherein 1) the one or more organoids from which the mono-dissociated mesenchymal cells are isolated comprise an intestinal tissue type, and 2) the organoid or enteroid from which the epithelial structure is isolated comprises a tissue type selected from an esophageal, gastric, hepatic, intestinal, or colonic tissue type, or any combination thereof.

9. The method of any one of the preceding claims, wherein the one or more organoids from which the mono-dissociated mesenchymal cells are isolated are small intestinal organoids, optionally human intestinal organoids (HIOs).

10. The method of any one of claims 7-9, wherein the tissue type of the one or more organoids from which the mono-dissociated mesenchymal cells are isolated and the tissue type of the organoid or enteroid from which the epithelial structure is isolated are different.

11. The method of claim 10, wherein the tissue type of the one or more organoids from which the mono-dissociated mesenchymal cells are isolated and the tissue type of the organoid or enteroid from which the epithelial structure is isolated are different, wherein no repatterning of the epithelial structure by the mono-dissociated mesenchymal cells occurs, such that the epithelium of the composite organoid maintains the tissue type of the organoid or enteroid,
optionally wherein the organoid or enteroid from which the epithelial structure is isolated is an organoid, and the organoid from which the epithelial structure is isolated is at least 14, 15, 16, 17, 18, 19, 20, 21, 22, 23, 24, 25, 26, 27, 28, 29, or 30 days old, or between 14-30, 15-30, 18-30, 15-20, or 15-25 days old;
or optionally wherein the organoid or enteroid from which the epithelial structure is isolated is an enteroid, wherein the enteroid is derived from adult tissue.

12. The method of claim 10, wherein the organoid or enteroid from which the epithelial structure is isolated is an organoid, wherein the tissue type of the one or more organoids from which the mono-dissociated mesenchymal cells and the tissue type of the organoid from which the epithelial structure is isolated are different, and wherein repatterning of the epithelial structure by the mono-dissociated mesenchymal cells occurs, such that the epithelium of the composite organoid exhibits properties of the tissue type of the one or more organoids, optionally wherein the organoid from which the epithelial structure is isolated is no more than 8, 9, 10, 11, 12, or 13 days old or between 8-13, 8-10, or 10-13 days old.

13. The method of any one of claims 7-9, wherein the tissue type of the one or more organoids from which the mono-dissociated mesenchymal cells are isolated and the tissue type of the organoid or enteroid from which the epithelial structure is isolated are the same.

14. The method of claim 13, wherein the one or more organoids and the organoid or enteroid each comprises only one of the esophageal, gastric, hepatic, intestinal, or colonic tissue type, such that the resultant composite organoid is a homogenous organoid comprising one tissue type.

15. The method of any preceding claim, wherein the one or more organoids is derived from pluripotent stem cells (PSCs) from a first subject, and the organoid or enteroid is derived from PSCs or isolated from gastrointestinal tissue from a second subject.

16. The method of claim 15, wherein the first subject and the second subject are mammals.

17. The method of claim 15 or 16, wherein the first subject and the second subject are humans.

18. The method of any one of claims 15-17, wherein the first subject and the second subject are the same individual.

19. The method of any one of the preceding claims, further comprising transplanting the composite organoid to a recipient subject.

20. The method of claim 19, wherein the recipient subject is the first subject or the second subject.

21. The method of claim 19 or 20, wherein the composite organoid exhibits greater engraftment and growth in the recipient subject compared to a comparable non-composite organoid or enteroid.

22. The method of any one of the preceding claims, wherein the 1) the one or more organoids from which the mono-dissociated mesenchymal cells are isolated or 2) the organoid or enteroid from which the epithelial structure is isolated, or both, are engineered to comprise one or more exogenous nucleic acids or proteins.

23. The method of any one of the preceding claims, wherein the 1) the one or more organoids from which the mono-dissociated mesenchymal cells are isolated or 2) the organoid or enteroid from which the epithelial structure is isolated, or both, comprise a genetic mutation, optionally wherein the genetic mutation is associated with a disease state or a model of a disease state.

24. A composite organoid, comprising:
a mesenchyme comprising mesenchymal cells isolated as mono-dissociated cells from a first organoid; and
an epithelium comprising epithelial cells isolated as an epithelial structure from a second organoid or enteroid.

25. The composite organoid of claim 24, wherein the ratio of the number of mesenchymal cells to epithelial cells in the composite organoid is greater than that of the second organoid or enteroid.

26. The composite organoid of claim 24 or 25, wherein the first organoid and second organoid or enteroid each comprises a tissue type selected from an esophageal, gastric, hepatic, intestinal, or colonic tissue type, or any combination thereof.

27. The composite organoid of claim 26, wherein the tissue type of the first organoid and the tissue type of the second organoid or enteroid is the same.

28. The composite organoid of claim 26, wherein the tissue type of the first organoid and the tissue type of the second organoid or enteroid is different.

29. The composite organoid of claim 28, wherein the tissue type of the first organoid from which the mesenchymal cells are isolated and the tissue type of the second organoid or enteroid from which the epithelial cells are isolated is different, wherein no repatterning of the epithelial cells by the mesenchymal cells occurs, such that the epithelium of the composite organoid maintains the tissue type of the second organoid or enteroid from which the epithelial cells are isolated,
optionally wherein the second organoid or enteroid is a second organoid, and the second organoid is at least 14, 15, 16, 17, 18, 19, 20, 21, 22, 23, 24, 25, 26, 27, 28, 29, or 30 days old or between 14-30, 15-30, 18-30, 15-20, or 15-25 days old; or
optionally wherein the second organoid or enteroid is a second enteroid, wherein the second enteroid is derived from adult tissue.

30. The composite organoid of claim 28, wherein the second organoid or enteroid is a second organoid, wherein the tissue type of the first organoid from which the mesenchymal cells are isolated and the tissue type of the second organoid from which the epithelial cells are isolated is different, and wherein repatterning of the epithelial cells by the mesenchymal cells occurs, such that the epithelium of the composite organoid exhibits properties of the tissue type of the first organoid from which the mesenchymal cells are isolated, optionally wherein the second organoid is no more than 8, 9, 10, 11, 12, or 13 days old or between 8-13, 8-10, or 10-13 days old.

31. A composite organoid, comprising:
a mesenchyme comprising a first tissue type selected from an esophageal, gastric, hepatic, intestinal, or colonic tissue type, or any combination thereof, and
an epithelium comprising a second tissue type selected from an esophageal, gastric, hepatic, intestinal, or colonic tissue type, or any combination thereof,
wherein the first tissue type and second tissue type have at least one difference in tissue types.

32. The composite organoid of any one of claims 24-31, comprising one or more exogenous nucleic acids or proteins.

33. The composite organoid of any one of claims 24-32, wherein the composite organoid has or is engineered to comprise a genetic mutation, optionally wherein the genetic mutation is associated with a disease state or a model of a disease state.

34. The organoid produced by the method of any one of claims 1-23.

35. The organoid of any one of claims 24-34 for use in treating a gastrointestinal malady in a subject in need thereof.

36. A method for screening for a candidate therapeutic, comprising contacting the organoid of any one of claims 24-34 with the candidate therapeutic and determining the effect of the candidate therapeutic on the organoid.

37. The method of claim 36, wherein the organoid is genetically modified, optionally genetically modified to exhibit a disease or model thereof.

38. The method of claim 36 or 37, wherein the mesenchyme and/or the epithelium of the organoid is genetically modified, optionally genetically modified to exhibit a disease or model thereof.
